(19) United States
(12) Patent Application Publication
(10) Pub. No.: US 2026/0273046 A1
(43) Pub. Date: Sep. 17, 2026

(54) METHOD OF VACCINATING FOR CANCER AND DEVICE AND KIT THEREFOR

(71) Applicant: Purdue Research Foundation, West Lafayette, IN (US)

(72) Inventors: Harm Hogenesch, West Lafayette, IN (US); Juan Francisco Hernandez-Franco, Hammond, IN (US)

(73) Assignee: Purdue Research Foundation, West Lafayette, IN (US)

(21) Appl. No.: 19/166,648

(22) PCT Filed: Apr. 1, 2024
(86) PCT No.: PCT/US2024/022525

§ 371 Date: Sep. 18, 2025

Related U.S. Application Data
(60) Provisional application No. 63/456,155, filed on Mar. 31, 2023.

(51) Int. Cl. A61K 39/385; A61K 9/00; A61K 39/00; A61P 37/04

(52) U.S. Cl. CPC A61K 39/385; A61K 9/0021; A61K 39/0011; A61K 39/0013; A61P 37/04; A61K 2039/545; A61K 2039/585

(57) ABSTRACT

A method of prophylactically or therapeutically vaccinating a subject for cancer comprising administering to the subject an immune response-inducing effective amount of a composition comprising (a) an agonist of the stimulator of interferon (IFN) genes (STING) and (b) a tumor (or cancer) antigen or neoantigen, both (a) and (b) of which are adsorbed onto cationic phytoglycogen (PG) nanoparticles; a microneedle device (e.g., microneedle patch) comprising a plurality of microneedles on/in which are contained cationic PG nanoparticles onto which are adsorbed (i) a STING agonist and (ii) a tumor (or cancer) antigen or a neoantigen; a needle-free injector comprising cationic PG nanoparticles onto which are adsorbed (i) a STING agonist and (ii) a tumor (or cancer) antigen or neoantigen; and a kit comprising (a) a microneedle device or a needle-free injector and (b) a composition comprising cationic PG nanoparticles onto which are adsorbed (i) a STING agonist and (ii) a tumor (or cancer) antigen or neoantigen.

### CROSS-REFERENCE TO RELATED APPLICATIONS

This application claims priority to U.S. provisional patent application No. 63/456,155, filed Mar. 31, 2023.

### TECHNICAL FIELD

The present disclosure relates to a method of vaccinating a subject for cancer, adjuvanted cancer vaccines, devices (e.g., microneedle injector, microneedle patch, and needle-free injectors), and kits.

### BACKGROUND

The last decade has seen significant advances in the research and development of therapeutic cancer vaccines. A therapeutic cancer vaccine must elicit tumor regression, prevent disease recurrence, and generate persistent antitumor memory, while limiting nonspecific and off-target responses. Natural antitumor immunity is dependent on the activation of antigen-presenting cells (APCs), such as dendritic cells (DCs), which capture and present tumor-secreted antigens to naïve T cells. Therefore, DCs are essential targets for cancer vaccines. To establish an effective tumor-specific T cell response, cancer vaccines must first stimulate and provide tumor antigen to DCs. Activated DCs migrate to secondary lymphoid organs, where they elicit tumor-specific CD4⁺ helper T cells and CD8⁺ cytotoxic T lymphocyte (CTL) responses.

Successful immunotherapy highly depends on CD8⁺ cytotoxic T lymphocytes (CTLs), which are critical components of the adaptive immune system's anticancer immunological responses. Currently, three immunotherapies have received FDA approval: immune checkpoint inhibitors that block CD8⁻ T cell suppression signaling pathways; tumor-specific CD8⁺ T cells with engineered chimeric antigen receptors (CARs); and a therapeutic vaccine, sipuleucel-T (Provenge), in which patient-derived DCs are loaded with prostate tumor antigen and injected back into the patient to activate tumor-specific CD8⁺ T cells. These immunotherapies have modernized cancer treatments, curing some patients and providing long-term remission for others; yet, some patients suffer recurrence, and the therapies are expensive.

Presently, the overall efficiency and cost-effectiveness of immunotherapies require further optimization. The need to expand the arsenal of cancer vaccines is underscored by the existence of just one FDA-approved vaccine compared to nine immune checkpoint inhibitors and six CAR-T cell therapies. Two recent clinical studies reveal significant induction of therapeutic immunity against stage III and IV melanomas in patients vaccinated with personalized cancer vaccines (Sahin et al., Nature 547: 222-226 (2017); Ott et al., Nature 547: 217-221 (2017)).

However, the vaccines elicited a CD4⁺ T cell response, rather than a CD8⁺ T cell response, in the majority of patients. The latter trial utilized poly-ICLC, an adjuvant that triggers immunological responses by binding to TLR-3, RIG-I, and MDA-5 and which, contrary to the clinical outcomes, was expected to elicit an IFN-γ⁺ Th1 and CD8⁺ T cell response. While these studies demonstrate the viability of adjuvanted vaccines in the treatment of cancer, they also emphasize the need for further research and development of adjuvants that can induce an anticancer CD8⁺ T cell response.

In view of the above, it is an object of the present disclosure to provide a method of vaccinating for cancer that can induce an anti-cancer CD8⁺ T cell response. This and other objects and advantages, as well as inventive features, will be apparent from the detailed description provided herein.

### SUMMARY

A method of prophylactically or therapeutically vaccinating a subject for cancer is provided. In one aspect, the method comprises administering to the subject an immune response-inducing effective amount of a composition comprising (or consisting essentially of or consisting of) an agonist of the stimulator of interferon (IFN) genes (STING) and a tumor (or cancer) antigen, both of which are adsorbed onto cationic phytoglycogen (PG) nanoparticles. The PG nanoparticles can be derived from sweet corn, such as sweet corn encoding the sugary-1 mutant gene. The cationic PG nanoparticles can be, and desirably are, prepared by conjugation of PG with octenyl succinic anhydride (OS) and (3-chloro-2-hydroxypropyl)-trimethylammonium chloride (CHPTAC) (PG-OS-CHPTAC (also known as Nano-11)).

The adsorption of the STING agonist onto cationic PG nanoparticles can, and desirably does, result in a synergistic effect. In examples, the synergistic effect includes an enhanced CD4+ and CD8+ T cell response via activation of NF-κB and IRF3 signaling pathways.

The STING agonist can be a cyclic dinucleotide (or analogue thereof) or a cyclic mononucleotide (or analogue thereof). The STING agonist can be ADU-S100 (also known as MIW815), a synthetic bisphosphorothioate analogue of 2′3′-c-di-AMP. The STING agonist can be a noncyclic dinucleotide (or analogue thereof) or a noncyclic mononucleotide (or analogue thereof). The STING agonist can be an amidobenzimidazole or a dimeric amidobenzimidazole. Other STING agonists include, but are not limited to, those selected from the group consisting of SYNB1891, MK-1454 (which is available from Merck), MK-2118, BMS-986301, SR-717, GSK3745417, SB-11285 (which is available from Spring Bank Pharmaceuticals), AdVCA0848, STINGVAX, IMSA-101, c-di-GMP (e.g., 2′3′-c-diGMP or halogenated c-di-GMP, such as fluorinated), c-di-AMP (e.g., 2′3′-c-di-AM(PS)2 (Rp, Rp) or halogenated c-di-AMP, such as fluorinated), cGAMP (e.g., 2′2′-cGAMP, 2′3′-cGAMP, 3′3′-cGAMP, 2′3′-cGAM(PS)2 (Rp/Sp), or halogenated 3′3′-cGAMP, such as fluorinated), cAIMP (e.g., cAIMP Difluor or cAIM(PS)2 Difluor (Rp/Sp)), cAMP-GMP, c-AMP-IMP, cGMP-IMP, 2′-5′, 3′-5′ mixed phosphodiester linkage (ML) RR-S2 c-di-AMP (also known as ML RR-S2 CDA), ML RR-S2-c-di-GMP (also known as ML-CDG), ML RR-S2 cGAMP, c-di-IMP, and vadimezan (also known as ASA-404 and DMXAA).

A tumor antigen consistent with the present disclosure can be a peptide, a protein, or a glycoprotein. The tumor antigen can be a tumor-associated antigen (TAA). In examples, the TAA is characterized by overexpression in tumor cells, i.e., an antigen that is expressed in normal cells but overexpressed in tumor cells. The tumor antigen can also be a tumor-specific antigen (TSA). In examples, the tumor antigen is a neoantigen. Such TSAs can be an antigen carrying an epitope or determinant constituting or incorporating a variant derived from a somatic mutation specific to a cancer or the cancer of an individual (i.e., a neoantigen). A TSA can also be an aberrantly expressed antigen, a self-antigen expressed from germline-specific genes activated in a cancer but not in normal tissues. These TSAs are often more widely shared among similar tumor types across individuals. The tumor antigen can be administered qua antigen or as an mRNA encoding the tumor antigen.

Examples of tumor antigens in melanomas include tyrosinase, MAGE (melanoma antigen gene)-A1, -C1, and -C2, Melan-A, Gp100, TRP-1, TRP-2, MC1R, NY-ESO-1, and SSX-2. Examples of lymphoma tumor antigens include CD19, CD20, CD30, CD79a, CD79b, and CCR4. Examples of prostate cancer antigens include prostate-specific antigen (PSA), prostatic acid phosphatase, TAG-72, and beta-catenin. Examples of more widely expressed TAAs include EphA3, Her2, and survivin.

Example tumor antigens include, but are not limited to, those selected from the group consisting of CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRylll); ganglioside G2 (GD2); ganglioside GD3 (aNeuSAc(2-8)aNeuSAc(2-3)bDGaip(1-4)bDGIcp(1-1)Cer); TNF receptor family member B cell maturation (BCMA); Tn antigen ((Tn Ag) or (GalNAcu-Ser/Thr)); prostate-specific membrane antigen (PSMA); Receptor tyrosine kinase-like orphan receptor 1 (RORI); Fms-Like, Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21(Testisin or PRSS21); vascular endothelial growth factor receptor 2 (VEGFR2); Lewis(Y)antigen; CD24; Platelet-derived growth factor receptor beta (PDGFR-beta); Stage-specificembryonic antigen-4 (SSEA-4); CD20; delta like 3 (DLL3); Folate receptor alpha;

Receptor tyrosine-protein kinase, ERBB2(Her2 /neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2(EphA2); Fucosyl GM1; sialyl Lewis adhesion molecule (sLe); ganglioside GM3 (aNeuSAc(2-3)bDGalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89), Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); and neoantigens.

Example tumor antigens also include, but are not limited to, those selected from the group consisting of CD150, 5T4, ActRIIA, B7, BMCA, CA-125, CCNA1, CD123, CD126, CD138, CD14, CD148, CD15, CD19, CD20,CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/nel, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-microglobulin, and Fc Receptor-like 5 (FcRL5).

The administering can be done via intradermal route. To that end, administering can involve the use of a microneedle injector, microneedle patch, or needle-free injector.

The cancer can be melanoma, lymphoma, or prostate cancer. Other cancers include, but are not limited to, bladder, breast, cervical, colon, colorectal, colorectal adenocarcinoma, gastroesophageal, gastrointestinal stromal, hepatocellular, kidney, leukemia, malignant mesothelioma, multiple myeloma, myelodysplastic syndrome, neuroblastoma, non-small cell lung, ovarian, pancreatic, plasma cell neoplasms, sarcoma, small cell lung, testicular, transitional cell carcinoma, urothelial, or Wilm's tumor.

Further provided is a microneedle device. The microneedle device comprises a plurality of microneedles on/in which are contained cationic phytoglycogen (PG) nanoparticles onto which are adsorbed (i) an agonist of the stimulator of interferon (IFN) genes (STING) and (ii) a tumor (or cancer) antigen or neoantigen. The microneedle device can be a patch. Also provided in this regard is a needle-free injector containing a composition comprising cationic PG nanoparticles onto which are adsorbed (i) an agonist of STING and (ii) a tumor (or cancer) antigen or neoantigen.

Even further provided is a kit. The kit comprises a microneedle device and a composition comprising (or consisting essentially of or consisting of) cationic PG nanoparticles onto which are adsorbed (i) an agonist of STING and (ii) a tumor (or cancer) antigen or neoantigen. Also provided in this regard is a kit comprising a needle-free injector and a composition comprising cationic PG nanoparticles onto which are adsorbed (i) an agonist of STING and (ii) a tumor (or cancer) antigen or neoantigen.

In examples, the composition contains a NanoST nanoparticle adjuvant comprising Nano-11 adjuvant, onto which is adsorbed an ADU-S100 STING agonist. As disclosed herein, NanoST produces strong synergistic immunostimulatory effects, viz., enhanced antigen cross-presentation and concomitant strong activation of NF-κB and IRF3 signaling pathways resulting in augmentation of the antigen-specific CD8+ cytotoxic T lymphocyte (CTL) response. To that end, in one example, A pharmaceutical composition for intradermal immunization against a cancer is provided, the composition comprising a NanoST nanoparticle adjuvant and a cancer antigen adsorbent. In examples, the cancer is a melanoma. In one such example, the cancer antigen is a tumor-associated antigen (TAA), characterized by overexpression in melanoma cells. Such cancer antigens can be, e.g., one of tyrosinase, MAGE (melanoma antigen gene)-A1, -C1, and -C2, Melan-A, Gp100, TRP-1, TRP-2, MC1R, NY-ESO-1, and SSX-2.

Relatedly, a method of immunizing a subject against a cancer is provided, which method comprises administering to the subject, via an intradermal route of administration, an immune response-inducing effective amount of a composition comprising a NanoST nanoparticle adjuvant, and a cancer antigen adsorbent, whereupon, through activation of NF-κB and IRF3 signaling pathways, the subject is immunized against the cancer. Administering can be performed by any suitable intradermal means that promotes localized, minimally-systemic delivery of the adsorbed cancer antigen. For example, intradermal administration may be performed using a microneedle injector, microneedle patch, or needle-free injector.

According one embodiment, the cancer is a melanoma. In examples, the cancer antigen is a tumor-associated antigen (TAA) characterized by overexpression in melanoma cells.5. For insteance, the TAA can be one of tyrosinase, MAGE (melanoma antigen gene)-A1, -C1, and -C2, Melan-A, Gp100, TRP-1, TRP-2, MC1R, NY-ESO-1, and SSX-2. In other examples, the cancer antigen is a tumor-specific antigen (TSA). By way of example, the TSA can be an epitope or determinant that constitutes or incorporates a mutation-derived biomarker specific to melanoma. an immunotherapeutic method is provided in which a subject is intradermally administered an immune response-inducing effective amount of the cancer vaccine, whereupon, through stimulatory activation of NF-κB and IRF3 signaling pathways, the subject is immunized against a cancer.

In another aspect, a method of administering a combination immunotherapy is provided. The combination immunotherapy method can include administration of a cancer vaccine of the present disclosure and co-administration of an immune checkpoint inhibitor. For instance, the method can involve (1) administering to a subject an immune response-inducing effective amount of a cancer vaccine comprising a cationic PG nanoparticle adjuvant onto which are adsorbed (i) an agonist of the stimulator of interferon (IFN) genes (STING) and (ii) a tumor (or cancer) antigen; and (2) co-administering to the subject a blockade-inducing effective amount of an immune checkpoint inhibitor, whereupon, the subject is immunized against a cancer.

In one example, the combination immunotherapy method can include steps of (1) administering to a subject an immune response-inducing effective amount of a cancer vaccine comprising a NanoST nanoparticle adjuvant and a cancer antigen adsorbent; and (2) co-administering to the subject a blockade-inducing effective amount of a PD-1 checkpoint inhibitor; whereupon, through stimulatory activation of NF-κB and IRF3 signaling pathways induced through administration of the cancer vaccine and a checkpoint protein blockade induced through co-administration of the immune checkpoint inhibitor, the subject is immunized against a cancer.

Respective administration and co-administration can be made according to an immunotherapy schedule. As an example, a preventative immunotherapy schedule can entail (1) administration of a first dose of the cancer vaccine on D1; (2) co-administration of the immune check point inhibitor according to a dosing regimen beginning any one of days D3-D6; and (3) administration of a second dose of the cancer vaccine between D14 and D21. As another example, a therapeutic immunotherapy schedule can entail (1) administration of a first dose of the cancer vaccine on a first day (D1); (2) co-administration of the PD-1 immune check point inhibitor according to a dosing regimen beginning on any one of days D3 or D4; and (3) administration of a second dose of the cancer vaccine between D5 and D8. In certain examples, the dosing regimen is a single dose regimen. In certain other examples, the dosing regimen is a multiple dose regimen.

### BRIEF DESCRIPTION OF THE FIGURES

FIGS. 1A and 1B depict flow cytometry analysis of respective CD80 and CD86 expression on ADU-S100-, Nano-11-, and NanoST-stimulated human THP-1 cells.

FIGS. 1C and 1D provide box and whisker plots depicting median fluorescence intensity (MFI) of respective CD80 and CD86 expression on ADU-S100-, Nano-11-, and NanoST-stimulated human THP-1 cells.

FIGS. 1E and 1F present respective measurements of optical density (OD) at 655 nm and relative light units (RLUs) to quantify NF-κB-SEAP activation and IRF-Lucia luciferase signaling, respectively, in human THP1-Dual™ cells stimulated with ADU-S100, Nano-11, and NanoST, where the bars represent the mean±SEM of three replicates.

FIG. 2A present photographs of superficial parotid lymph nodes 10 days after intradermal vaccination with OVA, Nano-11+OVA, ADU-S100+OVA, or NanoST+OVA, scale bar, 2 mm.

FIGS. 2B and 2C presents bivariate density plots and corresponding box and whisker plots displaying the frequencies and absolute numbers of respective GC B cells (B220⁺FAS⁺GL7⁺) and GC Tfh cells (CD3⁺CD4⁺CXCR5⁺PD-1⁺) 10 days following the second injection with the specified vaccines, where the bars of each corresponding bar graph represent the mean±SEM of three replicates

FIGS. 2D-2G present box and whisker plots of the median 25^{th}-75^{th} percentiles of respective OVA-specific antibody secreting cells (ASCs) (FIG. 2D) and serum anti-OVA (FIGS. 2E-G) titers after ID vaccination, in which four to six mice were used to generate the presented data for each group.

FIG. 2H provides bivariate density graphs demonstrating the CD4⁺ and CD8⁺ T cell frequencies of isolated splenocytes from mice that were OVA-restimulated for 24 hours.

FIG. 2I present box and whisker plots of quantified absolute numbers of IFN-γ⁺- and IL-17⁺-secreting CD4⁺ and IFN-γ CD8⁺ cells, where the bars represent the mean±SEM of three replicates.

FIG. 3A provides a box and whisker plot of B3Z cell activity, viz., the conversion of CPRG to β-galactosidase measured by optical density at 590 nm (OD 590), in DC2.4 cells primed with 10 μg/ml, 100 μg/ml, or 1000 μg/ml of OVA absorbed to Nano-11, where the bars represent the mean±SEM of four replicates.

FIG. 3B provides a box and whisker plot of B3Z cell activity, viz., the conversion of CPRG to β-galactosidase measured by optical density at 590 nm (OD 590), in DC2.4 cells primed with the combination NanoST adjuvant+10 μg/ml of OVA or 0.5 μg/ml SIINFEKL, Nano 11+10 μg/ml of OVA or 0.5 μg/ml SIINFEKL, and media as the positive and negative controls, respectively, where the bars represent the mean±SEM of four replicates.

FIG. 3C provides a box and whisker plot showing 24-hour progression of B3Z activity of DC2.4 cells primed with Nano-11+OVA or NanoST+OVA, where the bars represent the mean±SEM of four replicates.

FIG. 4A provides plotted flow cytometry analysis of lysed CFSE^{hi} donor splenocytes pulsed with SIINFEKL from the spleen and the vaccine injection site's superficial parotid draining lymph node in inoculated mice primed and boosted with OVA, Nano-11+OVA, or NanoST+OVA and a PBS control group.

FIG. 4B provides individual percentages of lysed target cells of the vaccinated mice based on calculated difference between the pulsed and control populations in the inoculated mice in comparison with PBS control group, where the bars represent the mean±SEM of four replicates, and ns indicates no statistical significance.

FIG. 5A shows the vaccine and T cell depletion schedule, treatments, and tumor volume mean (±SEM (n≥5)) at 10 days post-tumor (DPT), 20 DPT, and endpoint in C57BL/6 mice injected with 1×10⁶ B16-OVA/B16 and primed and boosted with OVA, Nano-11+OVA, NanoST+OVA, NanoST+OVA+αCD4, or NanoST+OVA+αCD8. and respective PBS control groups, where na (not available) indicates no animals reached DPT20.

FIGS. 5B-D present individual tumor growth curves (mm³) at 5 DPT increments (FIG. 5B), percent tumor-free (FIG. 5C), percent long-term overall survival (FIG. 5D) in C57BL/6 mice injected with B16-OVA, B16, where survival curves censored mice who died of reasons unrelated to tumor progression or that were still alive at the conclusion of the study and the log-rank (Mantel-Cox) curve comparison test was applied to determine the percentage of tumor-free mice and survival.

FIGS. 5E-H present corresponding tumor grown and survival curves data in C57BL/6 mice injected with 2.5×10⁶ E.G7-OVA and primed and boosted with OVA, Nano-11+OVA, NanoST+OVA, Nano-11+OVA+αCD8, and NanoST+OVA+αCD8, and respective PBS control groups.

FIGS. 6A-H present corresponding tumor grown and survival curves data in C57BL/6 mice injected with 1×10⁶ B16-OVA/B16 or 2.5×10^{6 E.G}7-OVA and primed and boosted with OVA, Nano-11+OVA, and NanoST+OVA.

FIGS. 7A-7D present results demonstrating that NanoST administered intradermally stimulates the expansion of SIINFEKL-specific effector CD8+ T cells. The intradermal vaccination of OVA, Nano-11+OVA, or NanoST+OVA was conducted on CD57BL/6J mice on day 1 and day1. The superficial parotid draining lymph node (dLN) located near the intradermal injection site, spleen, and bone marrow were collected on day 31 to quantify the CD3+ CD8+ effector T cell response with SIINFEKL-specificity. Bone marrow cells were isolated from the left femur and tibia of each mouse and screened for SIINFEKL+ CD8+ T cells. H-2Kb chicken OVA257-264 SIINFEKL tetramer was used to identify SIINFEKL-specific CD8+ T cells. Bivariate density plots display the frequencies and absolute numbers of effector CD8+ IFN-γ+ T cells, (FIG. 7A), SIINFEKL+ CD8+ T cells (FIG. 7B), SIINFEKL+ IFN-γ+ CD8+ T cells (FIG. 7C), bone marrow cells (FIG. 7D).

FIG. 8A-8H presents results of combination immunotherapy with NanoST-based cancer vaccines and PD-1 checkpoint blockade in mice and swine models. FIG. 8A presents the antigen-specific vaccination strategy, αPD-1 immunotherapy schedule, and tumor volume mean±SEM (n≥5) at indicated days post tumor (DPT) inoculation, in which C57BL/6J mice were inoculated in the right flank by subcutaneous (s. c.) injection with 3.5×105 B16-OVA cancer cells; na, not available, no animals reached DPT20. FIG. 8B-8C present respective individual tumor growth curves (mm³), percentage of tumor-free mice, and survival rate. FIG. 8E presents the whole cell lysate cancer vaccine schedule, αPD-1 immunotherapy regimen, and tumor volume mean±SEM (n≥5) at indicated days post tumor (DPT) inoculation, in which mice were subcutaneously inoculated with 3.5×105 B16 cancer cells. FIGS. 8F-8H present respective individual tumor growth curves (mm3), percentage of mice without palpable B16 tumors, and survival rate of mice. The log-rank (Mantel-Cox) test was used to evaluate the proportion of tumor-free mice and the survival rate.

### DETAILED DESCRIPTION

The present disclosure is predicated, at least in part, on the discovery of an adjuvant for cancer vaccines, e.g., prophylactic and therapeutic cancer vaccines. The adjuvant can induce activation of CD8+ T cells and their differentiation into cytotoxic T cells.

In certain embodiments, the adjuvant comprises ADU-S100 (MIW815, which is available from Aduro Biotech), which is a synthetic analogue with mixed phosphodiester linkages and a phosphorothioate backbone that is an agonist of the stimulator of interferon (IFN) genes (STING) and provides protection from in vivo degradation and superior affinity to all known human STING alleles (see Corrales et al., Cell Reports 11: 1018-1030 (2015)). Additionally, clinical trials have demonstrated the safety of ADU-S100 in human patients (see, e.g., Meric-Bernstam et al., Clinical Cancer Research 29: 110-121 (2023) (Meric-Bernstam, 2023); Meric-Bernstam et al., Clinical Cancer Research 28: 677-688 (2022) (Meric-Bernstam, 2022), each of which is incorporated herein by reference).

The other component of the adjuvant may be Nano-11, which demonstrates great efficacy in stimulating humoral and cell-mediated immunity in mice and pigs (see, e.g., Patil et al., J Nanobiotechnol 20: 539 (2022) (Patil, 2022); Hernandez-Franco et al., J. Immunol. 206 (4):700-711 (2021) (Hernandez-Franco, 2021); Lu et al., Journal of Controlled Release 204: 51-59 (2015) (Lu, 2015). Nano-11 is an adjuvant prepared from phytoglycogen (PG) collected from sweet corn by reacting the PG successively with octenyl succinic anhydride (OS) and (3-chloro-2-hydroxypropyl)-trimethylammonium chloride (CHPTAC) (see, e.g., U.S. Pat. No. 9,821,055, which is incorporated herein by reference). The synthesized nanoparticles have a diameter of approximately 70-80 nm and a positive surface charge (Lu, 2015).

Recent research has shown that the combination of the cyclic dinucleotide (CDN) cyclic-di-AMP (cdAMP), an agonist of STING, and Nano-11 has potent synergistic effects. (Hernandez-Franco, 2021). In particular, the Nano-11/cdAMP combination adjuvant stimulated an increased number of IFN-γ⁺ and IL-17⁺ CD4⁺ T cells (Th1 and Th17) and IFN-γ⁺ CD8⁻ T cells following intradermal immunization with ovalbumin (OVA).

Described herein is the combined use of ADU-S100 and Nano-11, referred to herein as NanoST nanoparticle adjuvant or, simply, NanoST. The NanoST nanoparticle adjuvant has been found to produce strong synergistic immunostimulatory effects, viz., enhanced antigen cross-presentation and concomitant strong activation of NF-κB and IRF3 signaling pathways resulting in augmentation of antigen-specific CD8+ cytotoxic T lymphocyte (CTL) response. The combination can be safely delivered intradermally to promote tumor-specific immunity and systemic protection against, e.g., melanoma and lymphoma cancers. The efficacy of NanoST, as demonstrated herein, provides strong support for its use as a novel adjuvant platform for the development and design of next-generation cancer vaccines and STING-target immunotherapies. The efficacy of NanoST is surprising and unexpected, given that in previous clinical trials, ADU-S100 was shown to have limited efficacy for solid tumors and lymphomas.

As demonstrated herein, NanoST enhanced the expression of CD80 and CD86 as well as the activation of the NF-κB and IRF3 signaling pathways in human THP-1 cells. NanoST facilitated the cross-presentation of OVA, as shown by the activation of SIINFEKL-specific B3Z CD8⁺ T cells in vitro and the target lysis of SIINFEKL-bearing splenocytes in vivo. Intradermal vaccination of mice with Nano-11 and NanoST prevented the development of tumors following subcutaneous injection of B16-OVA melanoma cells and E.G7-OVA lymphoma cells. The antitumor immunity was abolished by the depletion of CD8⁺ T cells but not by the depletion of CD4⁺ T cells. Therapeutic vaccination with Nano-11 and NanoST increased the survival rate of mice by inhibiting the growth of B16-OVA and E.G7-OVA-established tumors. These results provide a strong rationale for further development of NanoST as a safe and adaptable adjuvant platform for preventive cancer vaccines and immunotherapy by STING-targeted activation.

In view of the above, provided is a method of prophylactically or therapeutically vaccinating a subject for cancer. The method comprises administering to the subject an immune response-inducing effective amount of a composition comprising (or consisting essentially of or consisting of) (i) a STING agonist and (ii) a tumor (or cancer) antigen or a neoantigen, both of which are adsorbed onto cationic PG nanoparticles. The cationic PG nanoparticles can be, and desirably are, prepared by conjugation with octenyl succinic anhydride (OS) and (3-chloro-2-hydroxypropyl)-trimethylammonium chloride (CHPTAC) (PG-OS-CHPTAC (also known as Nano-11)) (see, e.g., U.S. Pat. No. 9,821,055, which is incorporated herein by reference). The adsorption of the STING agonist onto cationic PG nanoparticles results in a synergistic effect. The cationic PG nanoparticles can be derived from sweet corn, such as sweet corn encoding the sugary-1 mutant gene. Without intending to be bound by theory, it is believed that the cationic plant-derived nanoparticle helps to minimize the systemic diffusion of the agonist of STING from an injection site and helps to target adjuvant and antigen to dendritic cells.

The composition can be formulated as a pharmaceutical composition, which, in addition to (a) the STING agonist and (b) the tumor (or cancer) antigen or neoantigen, both of which are adsorbed onto cationic PG nanoparticles, can comprise (or consist essentially of or consist of) one or more carriers, excipients, or diluents. The pharmaceutical composition can be formulated using methods well-known in the art, including those described, for example, in Remington, the Science and Practice of Pharmacy, 23rd edition, Philadelphia, PA: Lippincott Williams and Wilkins, which is incorporated herein by reference.

“Carrier” can be used generically herein to refer to pharmaceutically acceptable carriers, diluents, other adjuvants, and excipients. In one example, about 80 μg (such as 80 μg) Nano-11 and about 5 μg (such as 5 μg) ADU-S100 are included in the composition.

As used herein, the term “vaccinating” encompasses prophylactic and therapeutic applications. Prophylactic vaccination prevents at least to some degree, and desirably to a high degree, including complete prevention, the onset of disease and/or slows the onset of disease.

As used herein, the term “administering” refers to a variety of parenteral and non-parenteral routes. Administration routes may be intratumoral or parenteral, including, but not limited to, one or more of subcutaneous, intravenous, intramuscular, intra-arterial, intradermal, intrathecal, and epidural administrations.

As used herein, a “subject” is a human or non-human animal. Thus, compositions, methods, and kits described herein may be applicable to human and veterinary applications.

As used herein, an “immune response-inducing effective amount” is an amount of a vaccine sufficient to induce an immune response in a subject to cancer. The amount may vary in view of a subject's age, sex, and weight.

As used herein, a “blockade-inducing effective amount” is an amount of an immune checkpoint inhibitor sufficient to induce an immune checkpoint blockade (ICB) suppressing T-cell regulation by checkpoint proteins expressed in a cancer. The amount may vary in view of a subject's age, sex, and weight.

An “agonist” herein, as it relates to a ligand and a receptor, may comprise a molecule, a combination of molecules, a complex, or a combination of reagents that bind the receptor.

The STING agonist may be any suitable STING agonist, such as a cyclic dinucleotide (or analogue thereof) or a cyclic mononucleotide (or analogue thereof). The STING agonist can be ADU-S100 (also known as MIW815), which is available from Aduro Biotech. The STING agonist can be a noncyclic dinucleotide (or analogue thereof) or a noncyclic mononucleotide (or analogue thereof). The STING agonist may be an amidobenzimidazole or a dimeric amidobenzimidazole. The STING agonist may be selected from the group consisting of SYNB1891 (Synlogic), MK-1454 (which is available from Merck), MK-2118, BMS-986301 (Bristol-Myers Squibb), SR-717, GSK3745417 (GlaxoSmithKline), SB-11285 (which is available from Spring Bank Pharmaceuticals), AdVCA0848, STINGVAX, IMSA-101 (ImmuneSensor Therapeutics), c-di-GMP (e.g., 2′3′-c-diGMP or halogenated c-di-GMP, such as fluorinated), c-di-AMP (e.g., 2′3′-c-di-AM(PS)2 (Rp, Rp) or halogenated c-di-AMP, such as fluorinated), cGAMP (e.g., 2′2′-cGAMP, 2′3′-cGAMP, 2′3′-cG^{S}A^{S}MP (also known as ADU-V16), 3′2′-cGAMP, 3′3′-cGAMP, 2′3′-cGAM(PS)2 (Rp/Sp), or halogenated 3′3′-cGAMP, such as fluorinated), cAIMP (e.g., cAIMP Difluor or cAIM(PS)2 Difluor (Rp/Sp)), cAMP-GMP, c-AMP-IMP, cGMP-IMP, 2′-5′, 3′-5′ mixed phosphodiester linkage (ML) RR-S2 c-di-AMP (also known as ML RR-S2 CDA), ML RR-S2-c-di-GMP (also known as ML-CDG), ML RR-S2 cGAMP, c-di-IMP, and vadimezan (also known as ASA-404 and DMXAA). Other STING agonists include, for example, CDK 002 (Codiak BioSciences), E7766 (Eisai), BI 1387446 (Boehringer Ingelheim), TAK-676 (Takeda), and SNX281 (Stingthera), FAA, DMXAA, CMA, G10, DSDP, α-mangostin, BMBC, diABZI Compound 3, MSA-2, SR-717, 2′-F-c-di-GMP, dithio-2′-F-cAIMP (see, e.g., Garland et al., Chem Rev 122: 5977-6039 (2022), which is hereby incorporated by reference for its teachings regarding same).

Cyclic purine dinucleotides include analogs, such as phosphorothioate analogues referred to as “thiophosphates.” Given that a phosphorothioate linkage is inherently chiral, the phosphates may independently exist in R or S form.

Other cyclic dinucleotide STING agonists include those disclosed in U.S. Pat. No. 11,401,295, USPAPN 2016/0287623, USPAPN 2017/0044206, USPAPN 2017/0146519, USPAPN 2017/0158724, USPAPN 2017/0283454, USPAPN 2019/0185509, USPAPN 2019/0185510, USPAPN 2019/0183917, USPAPN 2019/0322696, USPAPN 2019/0322697, USPAPN 2019/0359645, USPAPN 2021/0340169, Int'l Pat. App. Pub. No. WO 2018/118665, Int'l Pat. App. Pub. No. WO 2018/118664, Int'l Pat. App. Pub. No. WO 2018/100558, Int'l Pat. App. Pub. No. WO 2018/098203, Int'l Pat. App. Pub. No. WO 2018/067423, Int'l Pat. App. Pub. No. WO 2018/065360, Int'l Pat. App. Pub. No. WO 2018/060323, Int'l Pat. App. Pub. No. WO 2018/009466, Int'l Pat. App. Pub. No. 2017/075477, Int'l Pat. App. Pub. No. WO 2017/186711, Int'l Pat. App. Pub. No. WO 2017/161349, Int'l Pat. App. Pub. No. WO 2017/106740, and Int'l Pat. App. Pub. No. WO 2014/179760, each of which is incorporated herein by reference. Cyclic mononucleotide analogues, which are STING agonists, include those disclosed in USPAPN 2020/0131209, which is incorporated herein by reference. Other heterocyclic small molecule agonists are disclosed in Int'l Pat. App. Pub. No. WO 2018/234807, Int'l Pat. App. Pub. No. 2018/234805, and Int'l Pat. App. Pub. No. 2018/234808, each of which is incorporated herein by reference.

A tumor antigen consistent with the present disclosure can be a peptide, a protein, or a glycoprotein. The tumor antigen can be a tumor-associated antigen (TAA), an antigen that is expressed on normal cells but overexpressed in tumor cells. The tumor antigen can also be a tumor-specific antigen (TSA). Such TSAs can be an antigen carrying an epitope or determinant constituting or incorporating a variant derived from a somatic mutation specific to a cancer or the cancer of an individual. A TSA can also be an aberrantly expressed antigen, a self-antigen expressed from germline-specific genes activated in a cancer but not in normal tissues. These TSAs are often more widely shared among similar tumor types across individuals. The tumor antigen can be administered qua antigen or as an mRNA encoding the tumor antigen.

Examples of tumor antigens in melanomas include tyrosinase, MAGE (melanoma antigen gene)-A1, -C1, and -C2, Melan-A, Gp100, TRP-1, TRP-2, MC1R, NY-ESO-1, and SSX-2. Examples of lymphoma tumor antigens include CD19, CD20, CD30, CD79a, CD79b, and CCR4. Examples of prostate cancer antigens include prostate-specific antigen (PSA), prostatic acid phosphatase, TAG-72, and beta-catenin. Examples of more widely expressed TAAs include EphA3, Her2, and survivin.

In examples, the tumor antigen may be selected from the group consisting of CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRylll); ganglioside G2 (GD2); ganglioside GD3 (aNeuSAc(2-8)aNeuSAc(2-3)bDGaip(1-4)bDGIcp(1-1)Cer); TNF receptor family member B cell maturation (BCMA); Tn antigen ((Tn Ag) or (GalNAcu-Ser/Thr)); prostate-specific membrane antigen (PSMA); Receptor tyrosine kinase-like orphan receptor 1 (RORI); Fms-Like, Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21(Testisin or PRSS 21); vascular endothelial growth factor receptor 2 (VEGFR 2); Lewis(Y)antigen; CD24; Platelet-derived growth factor receptor beta (PDGFR-beta); Stage-specificembryonic antigen-4 (SSEA-4); CD20; delta like 3 (DLL3); Folate receptor alpha;

Receptor tyrosine-protein kinase, ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2(EphA2); Fucosyl GM1; sialyl Lewis adhesion molecule (sLe); ganglioside GM3 (aNeuSAc(2-3)bDGalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1), uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72;

Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); and neoantigens. The tumor antigen can be selected from the group consisting of CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-microglobulin, and Fc Receptor-like 5 (FcRL5).

Example neoantigens are disclosed in AU 2016/264623; U.S. Pat. No. 9,835,631; Int'l Pat App Pub No. WO 2017/205810; Int'l Pat App Pub No. WO 2016/172722; U.S. Pat. Nos. 9,238,064; 9,096,674; 8,580,529; and 8,394,385, each of which is incorporated herein by reference.

Administering may be by any suitable route as known in the art for the administration of vaccines, in particular cancer vaccines. For example, administering may be done intradermally, in which case the administering can involve the use of a microneedle injector, microneedle patch, or needle-free delivery device.

The cancer may be any cancer. By way of example, the cancer may be melanoma, lymphoma, or prostate cancer. In other examples, the cancer may be bladder, breast, cervical, colon, colorectal, colorectal adenocarcinoma, gastroesophageal, gastrointestinal stromal, hepatocellular, kidney, leukemia, malignant mesothelioma, multiple myeloma, myelodysplastic syndrome, neuroblastoma, non-small cell lung, ovarian, pancreatic, plasma cell neoplasms, sarcoma, small cell lung, testicular, transitional cell carcinoma, urothelial, or Wilm's tumor.

Methods described herein may be used in combination with additional cancer therapy. Examples of additional cancer therapies include surgery, radiotherapy, chemotherapy, toxin therapy, immunotherapy, cryotherapy, and gene therapy. In some instances, the cancer may be chemotherapy-resistant or radiotherapy-resistant. When combined with additional cancer therapy/therapies, the composition of the instant disclosure and any additional cancer therapeutic composition can be administered simultaneously or sequentially, in either/any order, by the same or different routes.

In view of the above, a microneedle device is provided. The microneedle device comprises a plurality of microneedles on/in which are contained cationic PG nanoparticles onto which are adsorbed (i) a STING agonist and (ii) a tumor (or cancer) antigen or a neoantigen.

The microneedle device can be a microneedle patch. Also provided in this regard is a needle-free injector (see, e.g., Ravi et al., Int J Pharm Investig 5(4): 192-199 (2015), which is incorporated herein by reference). The needle-free injector contains a composition comprising cationic PG nanoparticles onto which are adsorbed (i) a STING agonist and (ii) a tumor (or cancer) antigen or a neoantigen.

Further in view of the above, a kit is provided. The kit comprises a microneedle device and a composition comprising (or consisting essentially of or consisting of) cationic PG nanoparticles onto which are adsorbed (i) a STING agonist and (ii) a tumor (or cancer) antigen or a neoantigen. Also provided in this regard is a kit comprising a needle-free injector (see, e.g., Ravi et al. (2015), supra) and a composition comprising (or consisting essentially of or consisting of) cationic PG nanoparticles onto which are adsorbed (i) a STING agonist and (ii) a tumor (or cancer) antigen or a neoantigen.

In an example embodiment, an intradermal vaccine composition is provided in which the composition contains a Nano-11 adjuvant onto which is adsorbed (i) an ADU-S100 STING agonist; and (ii) a cancer antigen. As disclosed herein, the NanoST nanoparticle adjuvant produces strong synergistic immunostimulatory effects, viz., enhanced antigen cross-presentation and concomitant strong activation of NF-κB and IRF3 signaling pathways, resulting in augmentation of antigen-specific CD8⁺ cytotoxic T lymphocyte (CTL) response. The pharmaceutical composition can further contain one or more carriers suitable for intradermal administration of the NanoST as a biocarrier for localized delivery of the cancer antigen.

Relatedly, an immunotherapeutic method is provided in which a subject is intradermally administered an immune response-inducing effective amount of the NanoST-based vaccine composition, whereupon, through stimulatory activation of NF-κB and IRF3 signaling pathways, the subject is immunized against a cancer. The administering can involve the use of a microneedle injector, microneedle patch, or needle-free injector. The cancer can be any cancer amenable to immunotherapy via an intradermal route of administration. In particular examples, for instance, the cancer can be a melanoma, lymphoma or prostate cancer.

Dosage for each of the Nano-11 and ADU-S100 components of NanoST may vary depending on the subject and other factors. In human adult examples, Nano-11 may be ~0.25-4 mg/dose and ADU-S100 ~25-250 μg/dose. The NanoST vaccine may administered in one or more doses. In one example, a NanoST vaccine dose may contain 0.5 mg of Nano-11 and 50 μg of ADU-S100, where the vaccine is administered in two doses according to a vaccination schedule, in which the first dose is administered on a first day (D1) and the second dose is administered between D5 and D21. In therapeutic immunotherapy, the first dose is administered on a first day (D1) and the second dose is administered between D4 and D8, D5 and D8, or D5 and D10, and, in examples, may be D1 and D5, D1 and D6, D1 and D7, D1 and D8, D1 and D9, or D1 and D10. In examples of preventative immunotherapy, the first dose is administered on a first day (D1) and the second dose is administered between D12 and D21, D13 and D21, D14 and D21, or D14 and D24 and, in examples, may be D1 and D14, D1 and D15, D1 and D16, D1 and D17, D1 and D18, D1 and D19, D1 and D20, D1 and D21, D1 and D22, D1 and D23, or D1 and D24.

In another aspect, a method of administering a combination immunotherapy is provided. The combination immunotherapy method can include administration of a cancer vaccine of the present disclosure and co-administration of an immune checkpoint inhibitor. For instance, the method can involve (1) administering to a subject an immune response-inducing effective amount of a cancer vaccine comprising a cationic PG nanoparticle adjuvant onto which are adsorbed (i) an agonist of the stimulator of interferon (IFN) genes (STING) and (ii) a tumor (or cancer) antigen; and (2) co-administering to the subject a blockade-inducing effective amount of an immune checkpoint inhibitor, whereupon, the subject is immunized against a cancer. Example inhibitors include Programmed Death-1 (PD-1) inhibitors, PD Ligand-1 (PDL-1) inhibitors, and cytotoxic T lymphocyte associated protein 4 (CTLA-4) inhibitors.

In one example, the combination immunotherapy method can include steps of (1) administering to a subject an immune response-inducing effective amount of a cancer vaccine comprising a NanoST nanoparticle adjuvant and a cancer antigen adsorbent; and (2) co-administering to the subject a blockade-inducing effective amount of a PD-1 checkpoint inhibitor, whereupon, through stimulatory activation of NF-κB and IRF3 signaling pathways induced by administration of the vaccine composition and a checkpoint protein blockade induced through co-administration of the immune checkpoint inhibitor, the subject is immunized against a cancer. The PD-1 checkpoint inhibitor may be a monoclonal antibody or small molecule agent. In certain examples, the PD-1 checkpoint inhibitor may be selected from one or more FDA approved agents, e.g., nivolumab, pembrolizumab, and cemiplimab. The cancer vaccine and immune checkpoint inhibitor can be administered and co-administered respective as a single formulation or as separate formulations, by the same route of administration, or by separate routes, in single respective doses or in multiple doses.

Dosage for each of the Nano-11 and ADU-S100 components of NanoST as well as PD-1 checkpoint inhibitor may vary depending on the subject and other factors. In human adult examples, Nano-11 may be ~0.25-4 mg/dose and ADU-S100 ~25-250 μg/dose. Dosages for a PD-1 checkpoint inhibitor as contemplated herein may include indicated dosages for FDA approved agents.

Respective administration and co-administration can be made according to an immunotherapy schedule. PD-1 checkpoint inhibitor scheduling can be based on FDA-indicated dosing regiments for approved agents. In examples of therapeutic immunotherapy, the immunotherapy schedule may be: cancer vaccine doses, D1 and D5, PD-1 checkpoint inhibitor dosing regimen beginning D2-D4, cancer vaccine doses, D1 and D6, PD-1 checkpoint inhibitor dosing regimen beginning D2-D5, cancer vaccine doses, D1 and D7, PD-1 checkpoint inhibitor dosing regimen beginning D2-D6, cancer vaccine doses, D1 and D8 PD-1 checkpoint inhibitor dosing regimen beginning D2-D7, D1 and D9, PD-1 checkpoint inhibitor dosing regimen beginning D2-D6, cancer vaccine doses, or D1 and D6, PD-1 checkpoint inhibitor dosing regimen beginning D2-D9. In examples of preventative immunotherapy, the vaccination schedule of dosage administration may be cancer vaccine doses, D1 and D14, PD-1 checkpoint inhibitor dosing regimen beginning D2-D8, cancer vaccine doses, D1 and D15, PD-1 checkpoint inhibitor dosing regimen beginning D2-D8, cancer vaccine doses, D1 and cancer vaccine doses, D16, cancer vaccine doses, PD-1 checkpoint inhibitor dosing regimen beginning D2-D8, D1 and D17, cancer vaccine doses, PD-1 checkpoint inhibitor dosing regimen beginning D2-D8, D1 and D18, cancer vaccine doses, D1 and D19, PD-1 checkpoint inhibitor dosing regimen beginning D2-D8, cancer vaccine doses, D1 and D20, PD-1 checkpoint inhibitor dosing regimen beginning D2-D8, cancer vaccine doses, D1 and D21, cancer vaccine doses, D1 and D22, PD-1 checkpoint inhibitor dosing regimen beginning D2-D8, cancer vaccine doses, D1 and D23, or cancer vaccine doses, D1 and D24, PD-1 checkpoint inhibitor dosing regimen beginning D2-D8. In certain examples, the dosing regimen is a single dose regimen. In certain other examples, the dosing regimen is a multiple dose regimen.

As an example, a preventative immunotherapy schedule can entail (1) administration of a first dose of the cancer vaccine on D1; (2) co-administration of an immune check point inhibitor of a dosing regimen beginning D5; and (3) administration of a second dose of the cancer vaccine between D14 and D21. As another example, a therapeutic immunotherapy schedule can entail (1) administration of a first dose of the cancer vaccine on a first day (D1); (2) co-administration of a PD-1 immune check point inhibitor according to a dosing regimen beginning on D3; and (3) administration of a second dose of the cancer vaccine between D5 and D8.

### EXAMPLES

The following examples serve to illustrate the present disclosure. The examples are not intended to limit the scope of the claimed invention in any way.

Nano-11 was prepared as described in Lu, 2015. Briefly, in two sequential chemical processes, phytoglycogen (PG) nanoparticles from sweet corn encoding the sugary-1 mutant gene were conjugated with octenyl succinic anhydride (OS) and (3-chloro-2-hydroxypropyl)-trimethylammonium chloride (CHPTAC) to create PG-OS-CHPTAC (Nano-11). Endotoxin-free OVA was purchased from InvivoGen (San Diego, CA) and ADU-S100 from Chemitek (MIW815; Indianapolis, IN). Each reagent was resuspended in a sterile solution consisting of 10 mM Tris-saline (pH 7.4). Ultraperformance liquid chromatography/tandem mass spectrometry was utilized to quantify the adsorption efficiency of ADU-S100 onto Nano-11, as described in Patil, 2022. The ID vaccines were formulated by first combining 4 mg/ml Nano-11 with 250 μg/ml ADU-S100 to make the combination adjuvant (NanoST) for 1 hour at room temperature, followed by the adsorption of 500 μg/ml OVA to Nano-11, ADU-S100, or NanoST for 1 hour at room temperature.

For all experiments, six- to eight-week-old female C57BL/6J mice were purchased from the Jackson Laboratory (Bar Harbor, ME). They were housed in ventilated cages with ad libitum access to food and water, with three to four animals per box. The room was kept at 20±2° C. and 50±15% relative humidity, with a light/dark cycle of 12 hours. An acclimatization period of one week was given to the mice before the onset of the experiments. The Purdue University Animal Care and Use Committee gave approval to all animal experiments. Mice received 20 μl of vaccine through intradermal injection into the pinna of the ear. The vaccines were administered again after 7 or 21 days. Inhalational isoflurane was used to induce anesthesia in mice. Blood and tissue samples were collected 10 days after the second immunization, unless otherwise specified.

DC2.4 cells, which were cloned according to Shen et al., The Journal of Immunology 158: 2723-2730 (1997), which is incorporated herein by reference, were obtained from the Department of Pathology at the UMass Chan Medical School (Worcester MA). B3Z CD8⁺ T cell hybridoma, which were generated according to Karttunen et al., Proc. Natl. Acad. Sci. U.S.A. 89: 6020-6024 (1992), which is incorporated herein by reference, were obtained from the Department of Pathobiological Sciences at the University of Wisconsin School of Veterinary Medicine (Madison, WI). DC2.4 and B3Z cells were cultured in complete RPMI (RPMI 1640 supplemented with 2 mM L-glutamine, 55 μM beta-mercaptoethanol, 1×non-essential amino acids, 10 mM HEPES, 100 U/ml penicillin, 100 μg/ml streptomycin, and 0.25 μg/ml amphotericin) with 10% FBS. The B3Z cells were kept under selection by supplementing the culture media with 500 μg/ml geneticin (G418 sulfate, Thermo Fisher, MA). The B16-OVA melanoma cells that were utilized in the tumor studies were generously donated by Dr. Matthew Olson (Purdue University, IN). The OVA-expressing B16-OVA and EG.7-OVA cells were maintained in a selection environment with 0.4 mg/ml of G418 in RPMI 1640 supplemented with 2 mM L-glutamine, 55 μM beta-mercaptoethanol, 10 mM HEPES, 100 U/ml penicillin, 100 μg/ml streptomycin, and 0.25 μg/ml amphotericin, 4.5 g/L glucose, 1 mM sodium pyruvate, and 10% fetal bovine serum (FBS).

For preparation of cells for activation in vitro, human THP-1 cells (TIB-202; American Type Culture Collection Manassas, VA) were cultured in complete RPMI (RPMI 1640 supplemented with 2 mM L-glutamine, 55 μM β-mercaptoethanol, 1×non-essential amino acids, 10 mM HEPES, 100 U/ml penicillin, 100 μg/ml streptomycin, and 0.25 μg/ml amphotericin) with 10% FBS. Cells were seeded at a density of 5×10⁵ cells/well in a 96-well plate, stimulated with Nano-11 (80 μg/ml) and ADU-S100 (5 μg/ml) for 24 hours and 48 hours at 37° C. with 5% CO₂, and then processed for flow cytometry analysis. Similarly, the NF-κB-SEAP and IRF-Luc reporter THP1-Dual cells (InvivoGen, San Diego, CA) were grown at a density of 1×10⁵ cells/well in a 96-well plate in complete RPMI supplemented with 10% FBS, 100 μg/ml zeocin, 100 μg/ml normocin, and 10 μg/ml blasticidin. THP1-Dual cells were subsequently stimulated with Nano-11 (80 μg/ml) and ADU-S100 (5 μg/ml) for 24 hours and 48 hours at 37° C. with 5% CO₂, and the supernatants were collected for NF-κB and IRF pathway activation quantification.

The B3Z CD8⁺ T cell hybridoma cell line that expresses β-galactosidase under the control of the IL-2 promoter was utilized to detect antigen cross-presentation of the OVA 257-264 (SIINFEKL, InvivoGen, San Diego, CA) peptide (see Cruz et al., Annu. Rev. Immunol. 35: 149-176 (2017), which is incorporated herein by reference). Briefly, DC2.4 cells at 1×10⁵ cells/well on 96-well round bottom plates were cultured in complete RPMI containing 10% FBS. The DC2.4 cells were stimulated with 10, 100, or 1,000 μg/ml OVA adsorbed to 80 μg/ml Nano-11 or the NanoST nanoparticle adjuvant comprised of 80 μg/ml Nano-11 and 5 μg/ml ADU-S100 (NanoST) for 3 hours at 37° C. with 5% CO₂. Cells in media only or treated with 0.5 μg/ml OVA 257-264 (SIINFEKL) served as negative and positive controls, respectively. The cells were centrifuged at 300×g for 5 minutes and then washed three times with 200 μl of sterile PBS/0.1% BSA. Following fixation with 200 μl of phosphate-buffered saline (PBS)/0.2% paraformaldehyde for 15 minutes at 4° C., cells were washed twice with complete RPMI. Each well was seeded with 1×10⁵ B3Z cells in complete RPMI containing 10% FBS for 16 hours at 37° C. with 5% CO₂. Following centrifugation at 300×g for 5 minutes, cells were resuspended in 200 μl of lysis buffer containing 9 mM MgCl2, 0.125% NP40, and 0.15 mM chlorophenol red-β-D-galactopyranoside (CPRG, Sigma-Aldrich, St. Louis, MO). After 2, 4, 6, 8, 12, and 24 hours, the conversion of CPRG to β-galactosidase was quantified by measuring the absorbance at 590 nm (OD 590) using a microplate reader (BioTek Instruments, Winooski, VT).

For preparation of an antigen-specific cytotoxic T cell assay in vivo, splenocytes were isolated from naïve C57BL/6J mice and treated with red blood cell ACK lysis buffer to create a single-cell suspension. A total of 2×10⁷ cells/ml were pulsed with 1 M of SIINFEKL (pulsed) or left untreated (control) in HBSS and then incubated for 1 hour at 37° C. with 5% CO₂. The cells were centrifuged at 300×g for 5 minutes, resuspended in complete RPMI 10% FBS, centrifuged once more, and resuspended in PBS/0.1% BSA. SIINFEKL-pulsed cells at a concentration of 5×10⁶ cells/ml were stained with the fluorescent dye CFSE at 2.5 μM (CFSE^{hi}), whereas the control cells were stained with 0.25 μM (CFSE^{low}), immediately vortexed, and incubated for 10 minutes at 37° C. with 5% CO₂. A 5-minute centrifugation at 300×g was followed by a resuspension in complete RPMI with 10% FBS, additional centrifugation, and a final resuspension in PBS. Untreated cells and cells pulsed with SIINFEKL were combined at a 1:1 ratio to provide a final cell concentration of 2×10⁸ cells/ml. One hundred μl (1×10⁷ of each cell population) were injected into the tail vein of mice that had undergone two vaccinations with PBS, OVA, Nano-11+OVA, or NanoST+OVA, 21 days apart. The vaccinated mice were euthanized 4 hours later, and the spleen and draining lymph node near the injection site (superficial parotid) were isolated and processed into single cell suspensions. Target lysis of CFSE-labeled cells was quantified by flow cytometry. The total events corresponding to both fluorescent intensities (CFSE^{hi} and CFSE^{low}) were determined by flow cytometry. The percentage of lysis for each mouse was calculated as follows: % Lysis=100−[(Total CFSE^{hi} cells/Total CFSE^{low} cells)_{vaccinated mice}×100×(Total CFSE^{low} cells/Total CFSE^{hi} cells)_{PBS}].

The efficacy of preventive cancer vaccines was investigated by two intradermal injections with OVA, Nano-11+OVA, NanoST+OVA, or PBS with a 21-day interval. Eleven days following the second injection, mice were inoculated subcutaneously in the flank with 1.0×10⁶ B16-OVA, B16, or 2.5×10⁶ E.G7-OVA cells suspended in 100 μl of PBS. The tumor size was measured every other day, and the body weight was assessed to determine the body condition score using the scoring method described by Ullman-Culleré et al., Laboratory Animal Science 49 (1999), which is incorporated herein by reference. Using a caliper, the length (maximum longitudinal dimension) and width (maximum transverse dimension) of the subcutaneous tumor were measured in order to determine the approximate volume. Tumor volume was calculated using the ellipsoidal formula: V=½×(Length×Width²). Each tumor was measured by three observers to account for interobserver variability. The tumor endpoint measurements were taken when the tumors reached a volume of 2,000 mm³, when the tumors became ulcerated, when the body condition scores dropped below 2, or when the mice became moribund. Mice without palpable tumors 32 days after tumor inoculation were considered tumor-free and euthanized.

To mimic therapeutic immunotherapy, C57BL/6J mice were injected subcutaneously (s.c.) with 1.0×10⁶ B16-OVA or 2.5×10⁶ E.G7-OVA cells on day 0 to establish the tumor model. The B16-OVA-bearing mice received the same intradermal prophylactic vaccination (OVA, Nano-11+OVA, or NanoST+OVA) 8 days after tumor inoculation, followed by a second injection on day 15. The identical vaccination regimen was administered to E.G7-OVA-26 bearing mice at 1 and 21 days after tumor inoculation. The tumor endpoint measurements were recorded as described under prophylactic cancer vaccines. Mice without visible tumors were deemed tumor-free and euthanized 32 or 33 days post-tumor inoculation.

To evaluate the contribution of CD4⁺ and CD8⁺ T cells response to prophylactic-mediated vaccine protection against B16-OVA and E.G7-OVA tumor formation, C57BL/6J mice were vaccinated with OVA, Nano-11+OVA, or NanoST+OVA, and 21 days later the vaccination was repeated. Mice received 300 μg of anti-mouse CD4-depleting mAbs (clone GK1.5, Leinco, St. Louis, MO) or anti-mouse CD8α-depleting mAbs (clone 2.43, Leinco) 9 days after the second immunization through intraperitoneal (i.p.) injection and continued to receive 100 μg of αCD4 and αCD8α depletion treatment for 3 weeks on every fourth day. Two days following the initial depletion treatment, the flanks of mice were injected subcutaneously (s.c.) with 1.0×10⁶ B16-OVA or 2.5×10⁶ E.G7-OVA cells. The tumor growth and survival of mice were monitored for 33 days post-tumor inoculation.

OVA-specific IgG, IgG1, and IgG2c titers were measured by enzyme-linked immunosorbent assays (ELISA) on serum samples taken 10 days following the second vaccination, as previously reported (Hernandez-Franco (2021)). In short, 96-well plates were coated with 1 μg/ml OVA overnight at 4° C. After three washes with phosphate-buffered saline (PBS) containing 0.05% TWEEN (PBST; Sigma), the plates were blocked at room temperature (RT) for 2 hours with 200 μl PBST comprised of 1% BSA, followed by adding 100 μl of serially diluted serum samples to the wells in duplicate for 1 hour at 37° C. Wells were washed and incubated with 100 μl of peroxidase-conjugated goat anti-mouse IgG (1030-05), IgG1 (1073-05), or IgG2c (1079-05; SouthernBiotech, Birmingham, AL) for 1 hour at 37° C. Following a final wash, 100 μl of 3,3′,5,5′ tetramethylbenzidine substrate solution (Neogen, Lansing, MI) was added to all the wells and allowed to react in the dark for 10 min at RT. The reaction was stopped with the addition of 50 μl 2 M sulfuric acid, and absorbance at 450 nm (OD 450) was determined using the Synergy HT microplate reader (BioTek, Winooski, VT). End-point titers for OVA-specific antibodies were determined when the dilution at which OD 450 nm approached 0.2. The final OD values were obtained by subtracting the average value of three blank samples from the OD of the experimental test samples.

An ELISpot assay was performed to quantify the number of OVA-specific antibody-secreting cells (ASCs) in bone marrow cells extracted from the tibias and femurs of mice 10 days after the second vaccination, as previously described (Hernandez-Franco (2021)). Briefly, prior to euthanasia, MultiScreen IP filter plates (MAIPS4510; Millipore Sigma) were prepared by activating the filter membranes with 20 μl/well of 35% ethanol for 30 seconds and then washing the plates with 300 μl/well of sterile PBS three times. The wells were coated overnight at 4° C. with 10 μg/ml of OVA (100 μl/well) in sterile PBS. Wells were washed with sterile PBS and then blocked with complete RPMI (RPMI 1640 supplemented with 2 mM L-glutamine, 55 μM beta-mercaptoethanol, 1×non-essential amino acids, 10 mM HEPES, 100 U/ml penicillin, 100 μg/ml streptomycin, and 0.25 μg/ml amphotericin) with 10% FBS for 2 hours at 37° C. Followed by incubating the serially diluted single-cell suspensions of bone marrow cells in duplicate at 37° C. with 5% CO₂ for 24 hours. The plates were then washed five times with PBST and incubated at room temperature for two hours with biotin-labeled anti-mouse IgG (SouthernBiotech) in 1% FBS-containing PBST. This was followed by five washes, and then the plates were incubated with avidin-HRP conjugate (Thermo Fisher Scientific) in 1% FBS-containing PBST for 1 hour at 37° C. To initiate enzymatic activity, wells were exposed to 3-amino-9-ethylcarbazole (Sigma-Aldrich) treatment and protected from light for 10 minutes, followed by 15 washes with deionized water. The membrane tray was protected from light and dried at RT before the red-colored spots were quantified utilizing an ELISPOT reader (AID Diagnostika, Strassberg, Germany).

For flow cytometry analysis, single-cell suspensions were prepared from isolated splenocytes or lymph nodes and washed with Cell Staining Buffer (CSB; BioLegend, San Diego, CA). The staining protocols were conducted in accordance with the manufacturer's instructions, and unless otherwise specified, all mAbs were obtained from BioLegend. Live, viable cells were treated for 30 minutes at 4° C. with an anti-mouse CD16/32 (clone 93) antibody. Cells were labeled with anti-mouse mAbs against CD3ε (clone 145-2C11), CD4 (clone GK 1.5), CD8α (clone 53-6.7), CD185/CXCR5 (clone L138D7), CD279/PD-1 (clone 29F.1A12), GL-7 (clone GL7), B220 (clone RA3-6B2), and CD95/FAS (clone SA367H8) in CSB for 45 minutes at 4° C. In order to perform intracellular cytokine staining (ICS), splenocytes were first cultured in complete RPMI supplemented with 10% FBS and 25 μg/ml OVA for 24 hours at 37° C. with 5% CO₂. Thereafter, the cells were stimulated at 37° C. with 5% CO₂ for 6 hours with PMA, ionomycin, and monensin in complete RPMI supplemented with 10% FBS. Subsequently, the splenocytes were labeled with mAbs following the same method as described above and then permeabilized using Perm Wash Buffer (BioLegend) in preparation for ICS with IFN-γ (clone XMG1.2) and IL-17A (clone TC11-18H10.1). The cells were washed with CSB prior to being fixed using a 4% paraformaldehyde fixation buffer (BioLegend). Human THP-1 cells were labeled with anti-human CD11b (clone ICRF44), anti-human CD80 (clone 2D10), and anti-human CD86 (clone BU63) mAbs after 30 minutes of treatment with human TruStain FcX (BioLegend) at 4° C. Flow cytometry was conducted using an Attune NxT flow cytometer (Invitrogen, Waltham, MA), and the data were analyzed using FlowJo software (FlowJo, Ashland, OR).

To identify statistically significant differences between experimental groups, a one- or two-way analysis of variance (ANOVA) test was conducted, followed by Tukey's or Sidak's multiple comparison test. The percentage of tumor-free mice and survival rate were determined using the log-rank (Mantel-Cox) curve comparison test. All statistics were performed using GraphPad Prism (version 9.2, San Diego, CA). The results are presented as mean±SEM. A value of p<0.05 indicated statistical significance (*p<0.05, **p<0.01, ***p<0.001, and ****p<0.0001).

### Example 1—Nanost Combination Adjuvant Synergy on Costimulatory Molecules and Cell Signaling Expression

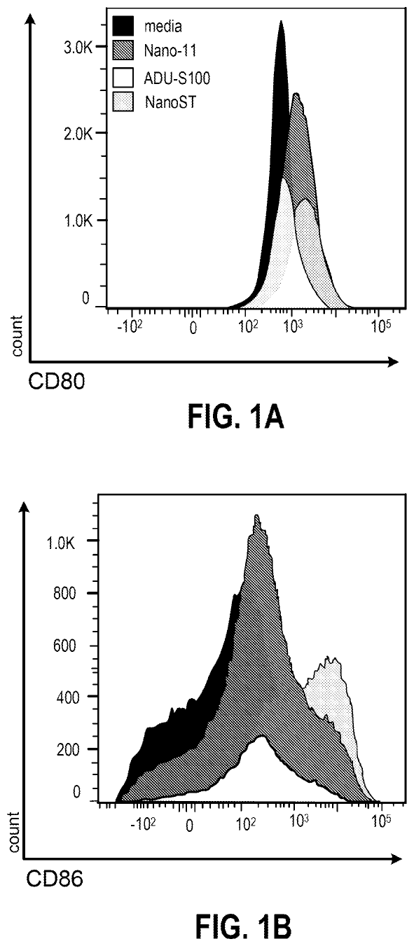

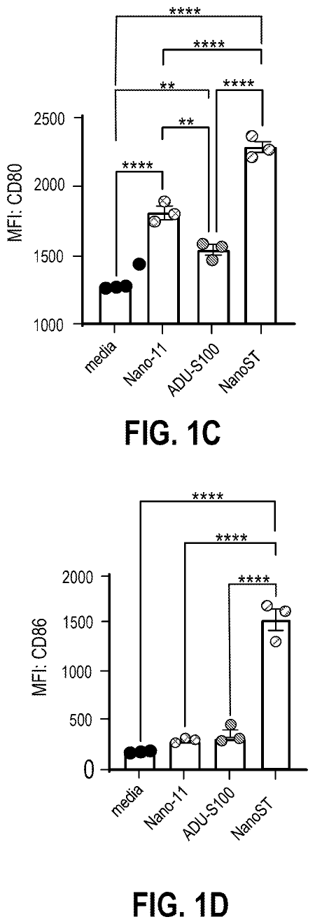

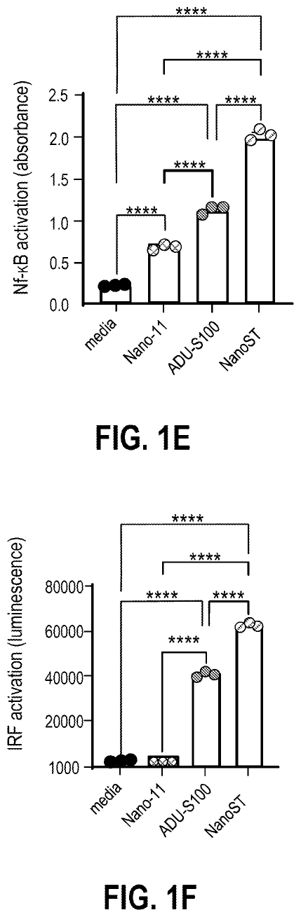

Recently it has been demonstrated that combining Nano-11 with cdAMP produced a synergistic and enhanced expression of MHC class II as well as the immunostimulatory CD80 and CD86 molecules in human, murine, and swine monocyte-derived DCs (Hernandez-Franco, 2021). To determine if Nano-11 synergizes with ADU-S100 to elicit an immunological response, the ability of the combination of Nano-11 and ADU-S100 (NanoST) to enhance DC activation was tested. As demonstrated in FIGS. 1A and 1C, Nano-11 and ADU-S100 alone induced CD80 expression in human THP-1 cells, while stimulation with the NanoST combination adjuvant further enhanced CD80 expression. Similarly, with reference to FIGS. 1B and 1D, NanoST stimulation resulted in significantly higher CD86 expression in THP-1 cells compared to cells treated with Nano-11 or ADU-S100 alone, neither of which had any effect on CD86 expression.

Furthermore, to examine the activation of NF-κB and IRF signaling pathways by Nano-11, ADU-S100, and NanoST, the NF-κB-SEAP and IRF-Lucia luciferase THP1-Dual reporter cell line was used. Stimulation with ADU-S100 alone activated NF-κB and IRF3 in THP-1 Dual cells. As demonstrated in FIGS. 1E and 1F, while Nano-11 promoted NF-κB activation but not IRF3 activation, the NanoST combination adjuvant produced a significant synergistic effect and elicited substantially higher activation of both signaling pathways. Together, these studies confirm that the combination of Nano-11 and ADU-S100 had marked synergistic effects on the activation of antigen-presenting cells.

### Example 2—Intradermal Immunization with NanoST Primes an Effective Systemic Cell-Mediated and Humoral Immune Response

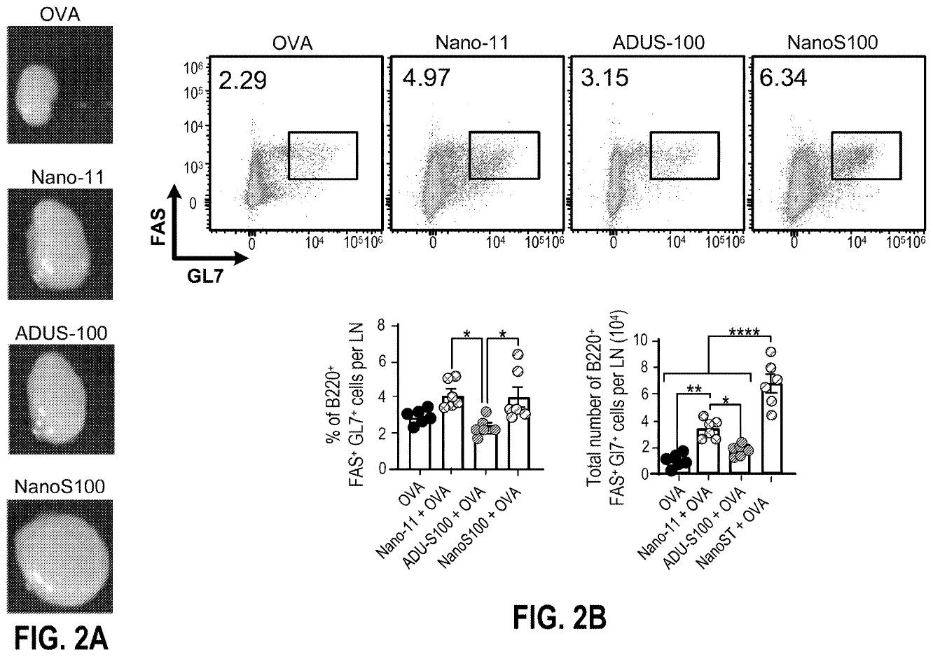

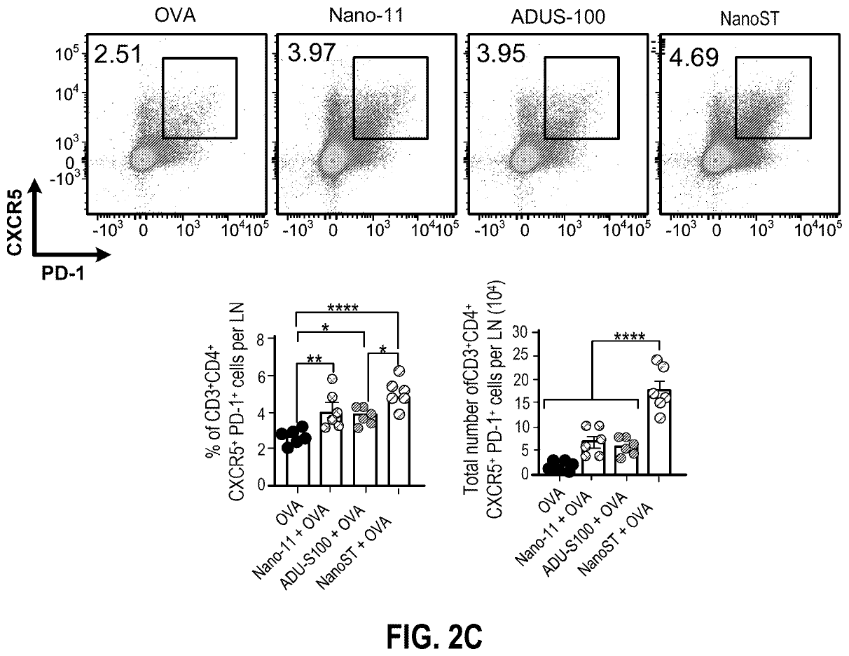

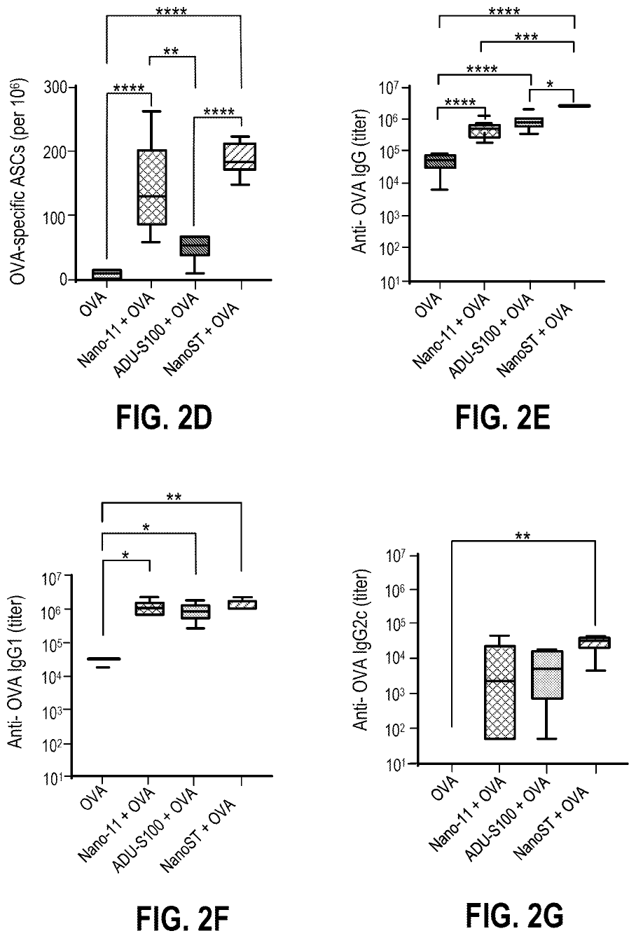

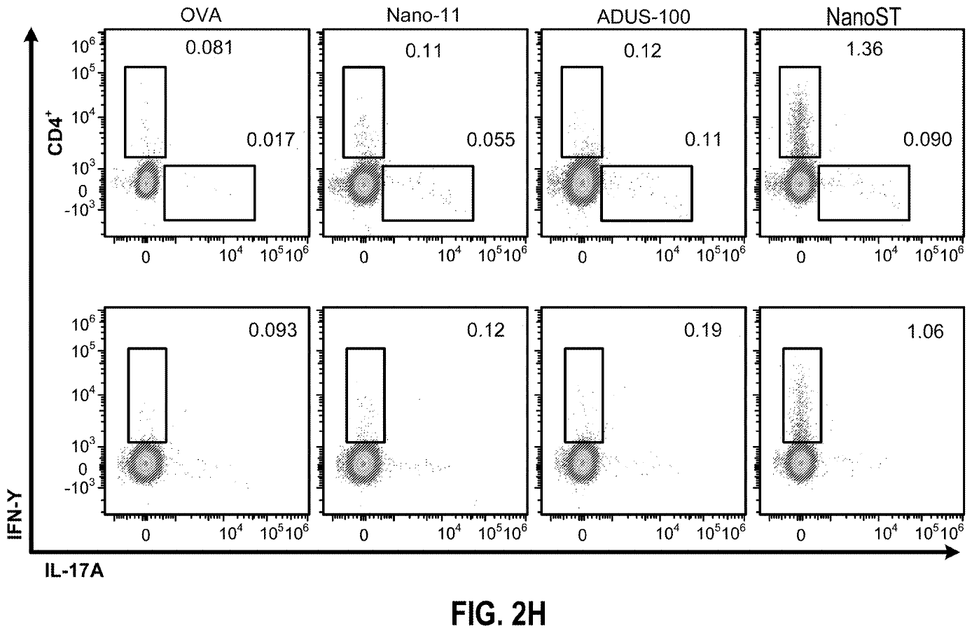

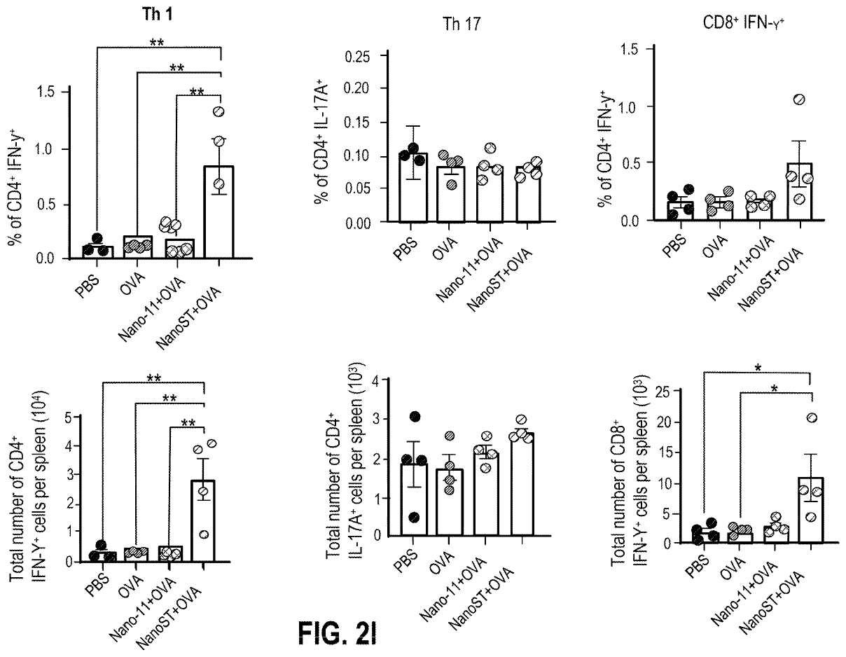

To investigate the immunogenicity of the combination adjuvant NanoST, composed of Nano-11 and ADU-S100, mice were intradermally immunized with OVA alone, OVA with Nano-11, OVA with ADU-S100, or OVA with NanoST. With reference to ig. 2A, Mice vaccinated with Nano-11, ADU-S100, or NanoST had an enlarged draining lymph node (dLN; superficial parotid) 10 days after the second intradermal (i.d.) injection of the ear pinna. As illustrated in FIGS. 2B and 2C, flow cytometric analysis was performed on the dLNs to quantify the number of respective GC B cells and follicular helper T (Tfh) cells. Nano-11 and NanoST induced a significant increase in the proportion and number of GC B cells; however, only the NanoST combination adjuvant promoted an increase in both the percentage and number of Tfh cells. As demonstrated in FIG. 2D, Nano-11 alone induced the differentiation of antigen-specific antibody secreting cells (plasma cells) in the bone marrow, which was significantly enhanced when Nano-11 and ADU-S100 were combined. With reference to FIGS. 2E and 2F, each of Nano-11 and ADU-S100 alone enhanced anti-OVA total IgG and IgG1 titers. However, as FIG. 2G demonstrates, the NanoST combination adjuvant significantly increased both titers in addition to inducing the production of IgG2c.

The number of antigen-specific effector CD4⁺ and CD8⁺ T cells was evaluated by intracellular cytokine detection following re-stimulation of splenocytes with OVA. As FIGS. 2H and 2I demonstrate, a marked increase of CD4⁺ IFN-γ⁺ and CD8⁺ IFN-γ⁺ T cells was detected in the spleen of mice immunized with NanoST compared to mice vaccinated with Nano-11 or ADU-S100 alone. Indeed, with specific reference to FIG. 2I, only mice vaccinated with NanoST had a significant increase in frequency and total number of OVA-specific IFN-y-secreting CD4⁺ and CD8⁺ T cells. Thus, the intradermal immunization with the NanoST combination adjuvant elicited a robust antigen-specific humoral and cell-mediated immune response. The significant activation of OVA-specific CD8⁺ IFN-γ⁻ T cells after intradermal vaccination with NanoST is an indication of the cross-presentation of OVA-derived peptides.

### Example 3—DCs Primed with Nano-11 or NanoST Cross-Present and Activate Antigen-Specific CD8⁺ T Cells

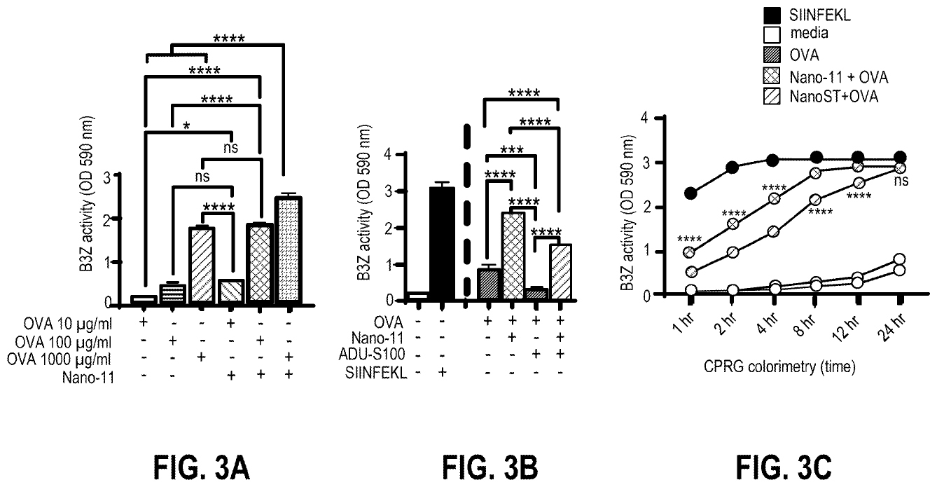

Murine DC2.4 dendritic cells were recruited to determine in vitro the capacity of Nano-11 and NanoST to promote the cross-presentation of OVA to CD8⁺ T cells. The DC2.4 cells were first stimulated with Nano-11 and increasing concentrations of OVA, followed by co-culture with the OVA 257-264 (SIINFEKL)-specific B3Z lacZ inducible CD8⁺ T cell hybridoma line. As demonstrated in FIG. 3A, the cross-presentation of OVA and activation of B3Z CD8⁺ T cells by DC2.4 cells were significantly enhanced when OVA was adsorbed to Nano-11. In contrast, as FIG. 3B shows, ADU-S100 decreased the cross-presentation of OVA when added alone and reduced the stimulatory effect of Nano-11 when added together with Nano-11. However, with reference to FIG. 3C, extending the reaction time of NanoST+OVA yielded the same B3Z activation capacity as Nano-11. Nevertheless, both Nano-11+OVA and NanoST+OVA stimulated DC2.4s significantly, which induced SIINFEKL-specific B3Z CD8⁺ T cell activation compared to unadjuvanted-OVA.

### Example 4—Vaccination with NanoST Induced Antigen-Specific Lysis of Target Cells

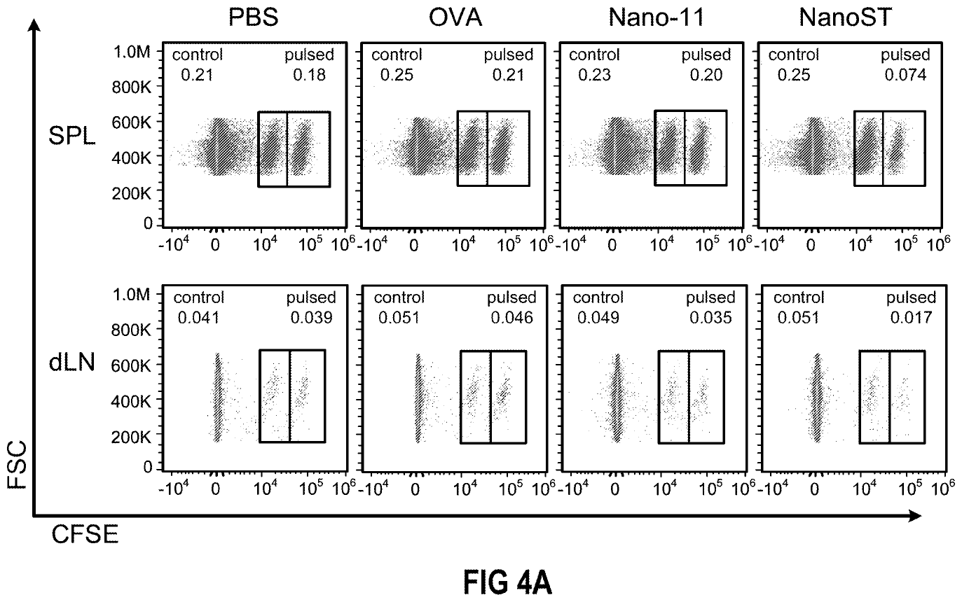

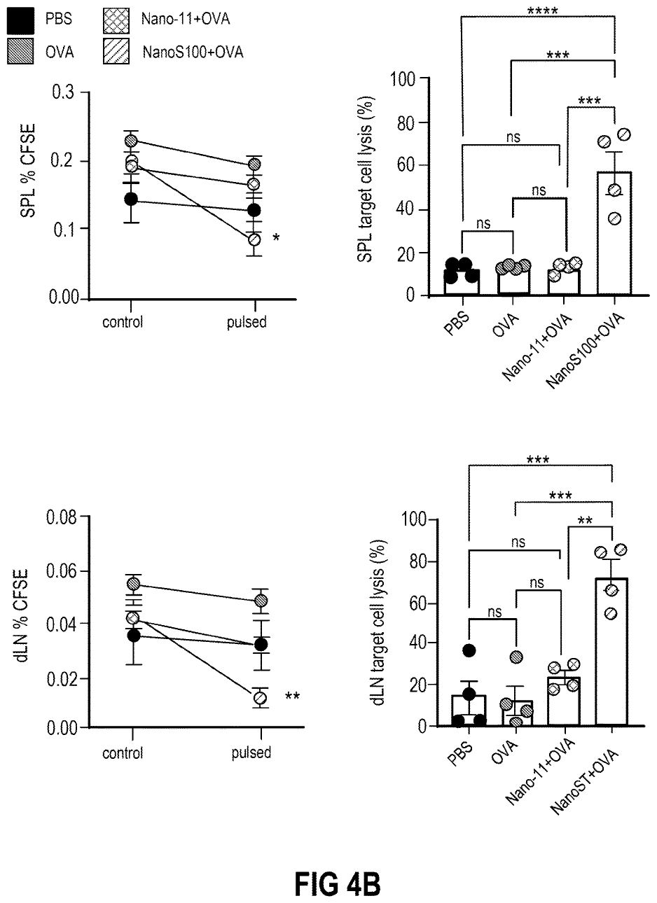

Nano-11 and NanoST were determined to promote OVA cross-presentation in DC2.4 cells in vitro, leading to the presentation of OVA peptides through MHC class I to the SIINFEKL-specific B3Z CD8⁺ T cells. To establish whether Nano-11 and NanoST facilitated the cross-presentation of OVA in vivo, donor CFSE+SIINFEKL-labeled splenocytes were intravenously (i.v.) injected into mice 10 days after administration of the booster vaccine. Referring to FIG. 4A, bivariate density plots of isolated cells from the spleen and dLN of mice immunized with NanoST+OVA showed a reduction in the percentage of CFSE signal in SIINFEKL-pulsed donor cells. In the spleen and dLN of mice immunized with NanoST+OVA, the number of CFSE^{hi} cells pulsed with the SIINFEKL peptide is significantly lower than the number of CFSE^{low} cells pulsed without the peptide compared to OVA and Nano-11+OVA. Quantitative analysis of the proportion of lysed target cells in mice immunized with the NanoST combination adjuvant as shown in FIG. 4B suggested that NanoST, but not Nano-11, significantly enhanced the in vivo cross-priming of antigen-specific cytotoxic CD8⁺ T lymphocytes. These results led us to evaluate the efficacy of Nano-11 and NanoST as adjuvant platforms for STING-targeted cancer immunotherapy.

### Example 5—Intradermal Vaccination with Nano-11 and NanoST Prevents the Growth of Tumors

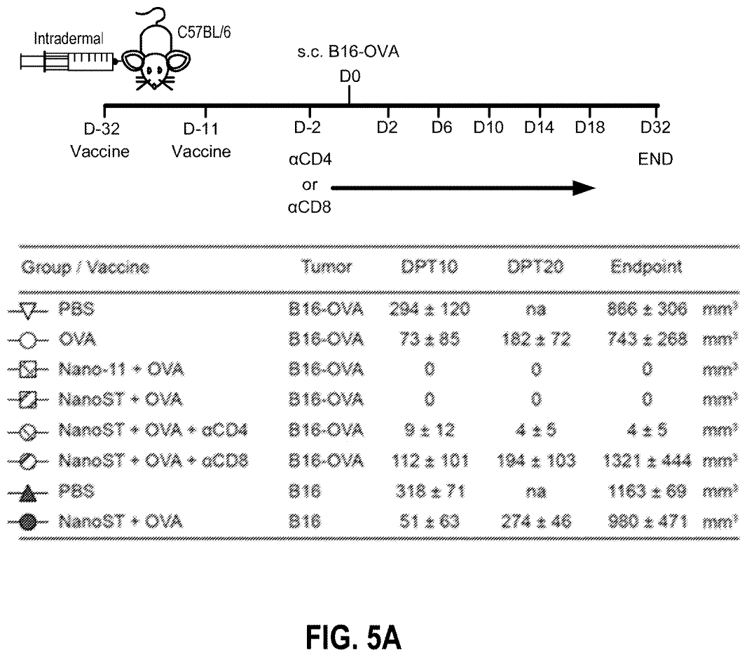

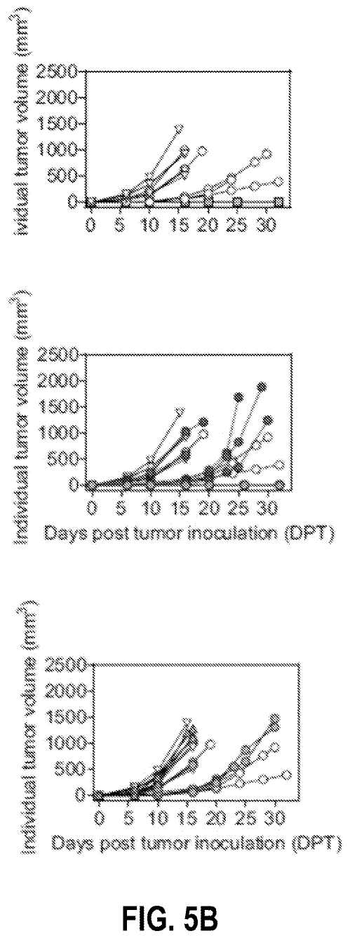

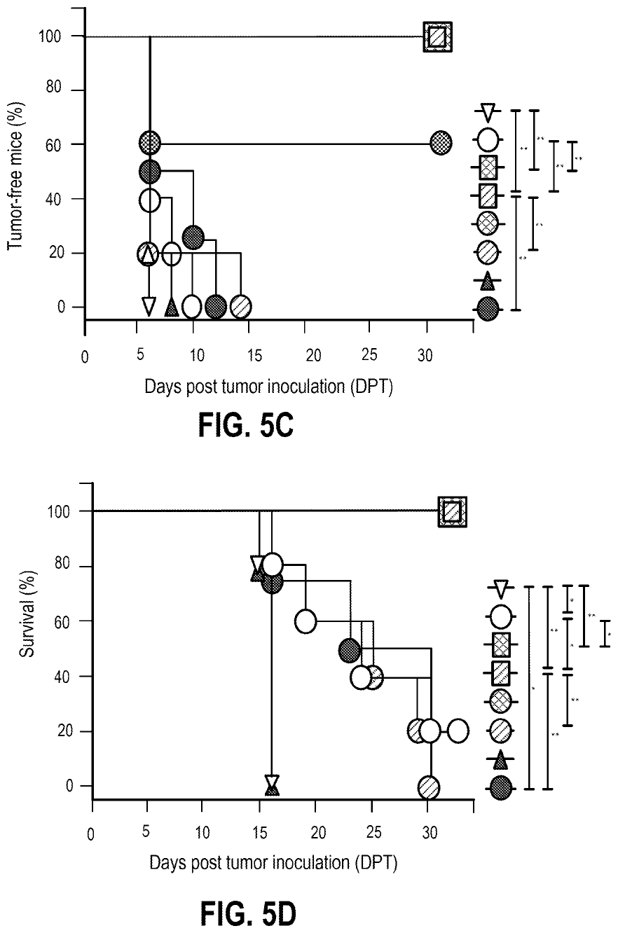

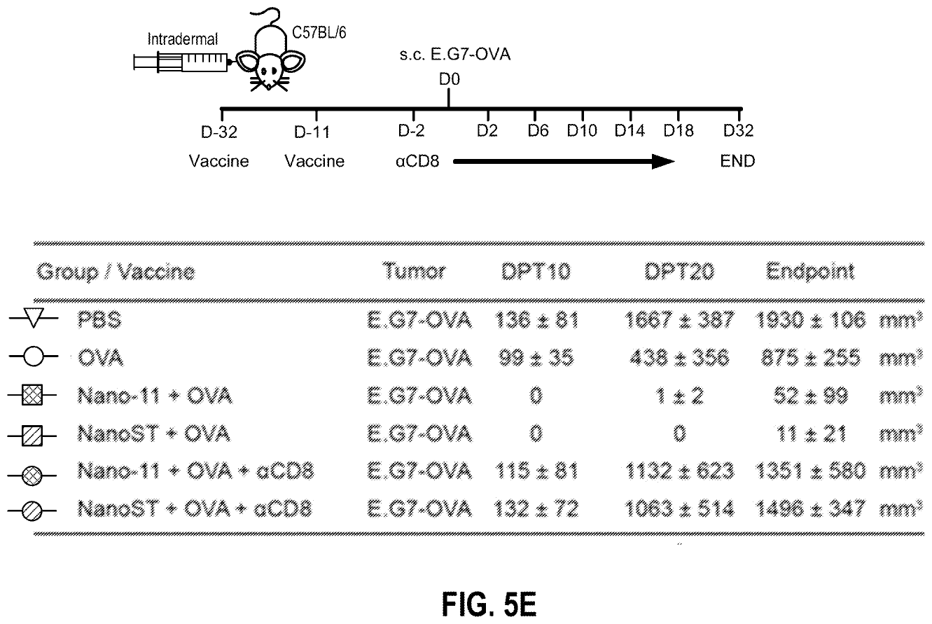

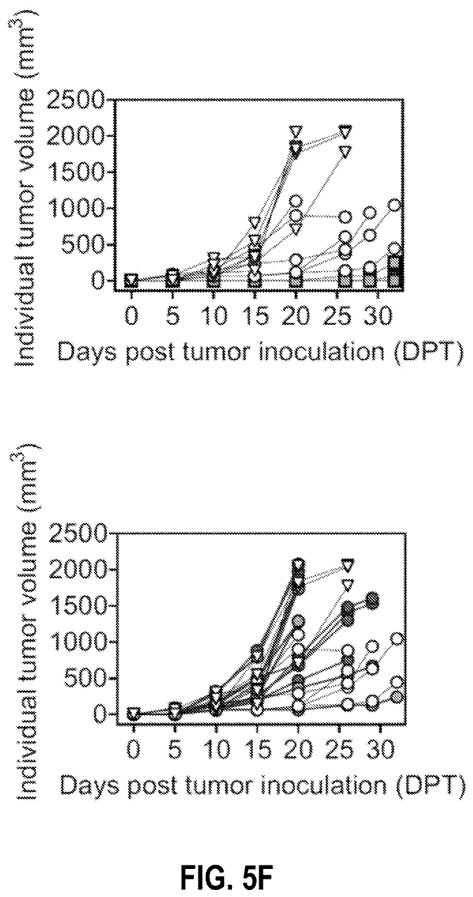

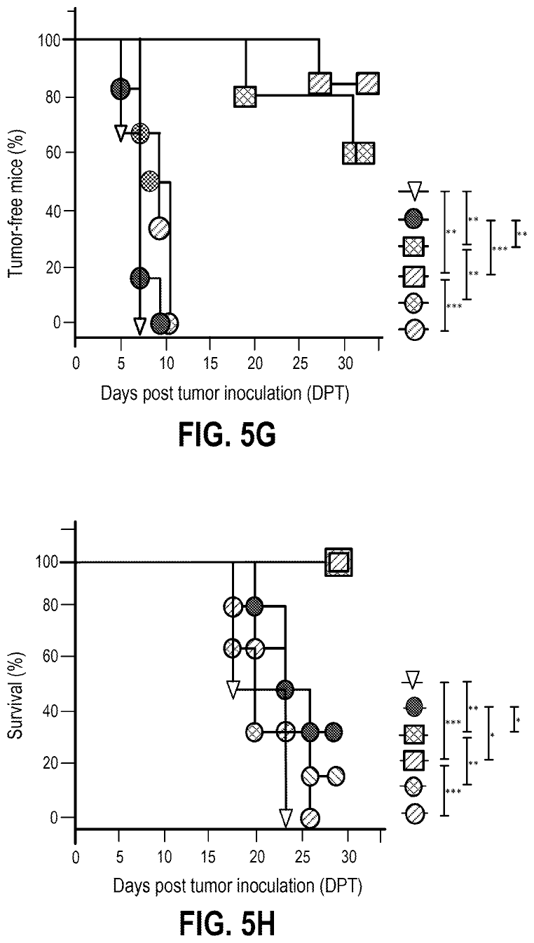

The immunotherapy properties of Nano-11 and NanoST in murine models of melanoma and lymphoma were investigated, in which OVA functions as a tumor-specific antigen. Mice were immunized twice intradermally with a three-week interval, followed by subcutaneous inoculation with B16, B16-OVA (FIG. 5A), or E.G7-OVA (FIG. 5E) tumor cells. In some experimental groups, CD4⁺ or CD8⁺ T cells were depleted 2 days before and every 4th day after cancer cell inoculation for 3 weeks. By day 10, all the PBS- and OVA-vaccinated control mice were positive for tumors. As illustrated in FIG. 5B, the immunization delayed, but did not prevent, the progression of tumor growth in B16 melanoma cells. In contrast, as FIG. 5C shows, mice immunized with Nano-11+OVA or NanoST+OVA were completely protected against the development of tumors when inoculated with B16-OVA melanoma cells. Referring to FIGS. 5F and 5G, although a few mice immunized with Nano-11+OVA or NanoST+OVA developed E.G7-OVA lymphomas, the tumors in these mice were small, and the majority of mice remained tumor-free. As shown in FIGS. 5D and 5H, all mice immunized with Nano-11 and NanoST survived the 32-day observation period after inoculation with B16-OVA or E.G7-OVA.

Depletion of CD8⁺ T cells abolished the protection against B16-OVA and E.G7-OVA elicited by Nano-11 or NanoST. In contrast, depletion of CD4⁺ T cells had no significant effect on the preventative efficacy of NanoST. These results indicate that the antitumor protection induced by Nano-11 and NanoST adjuvants was dependent on the activation and expansion of tumor-specific CTLs.

### Example 6—Therapeutic Immunity from Intradermal Vaccination with Nano-11 and NanoST

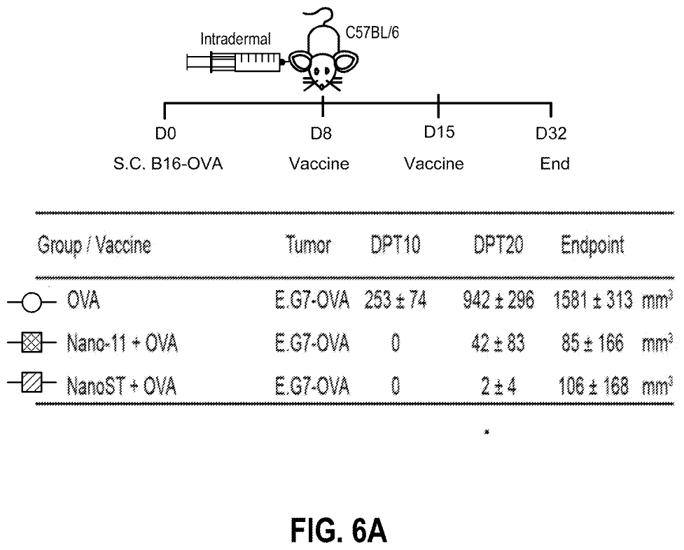

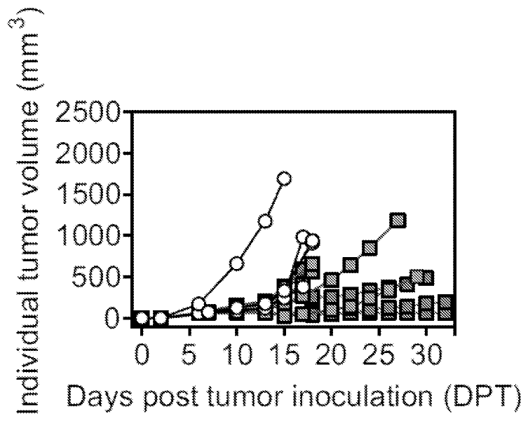

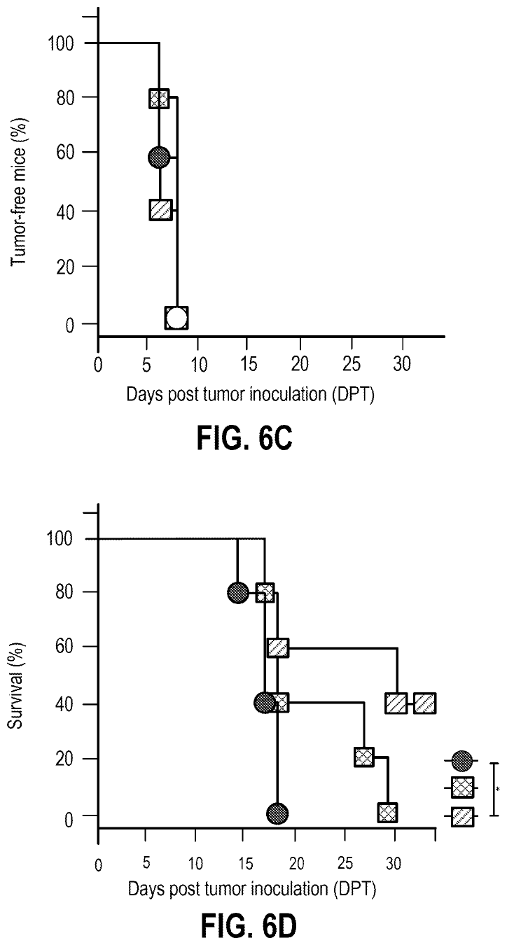

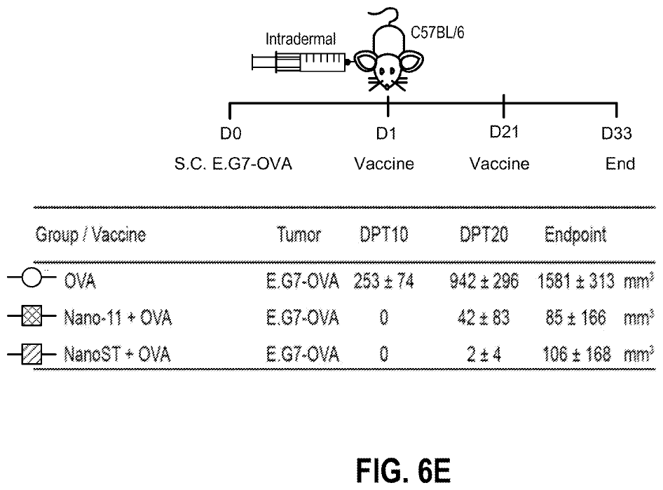

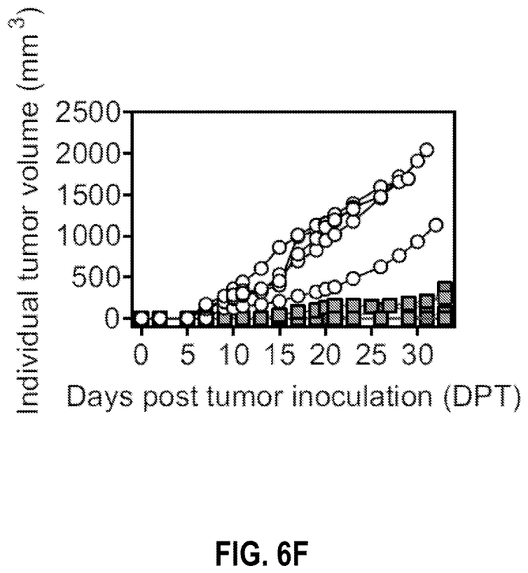

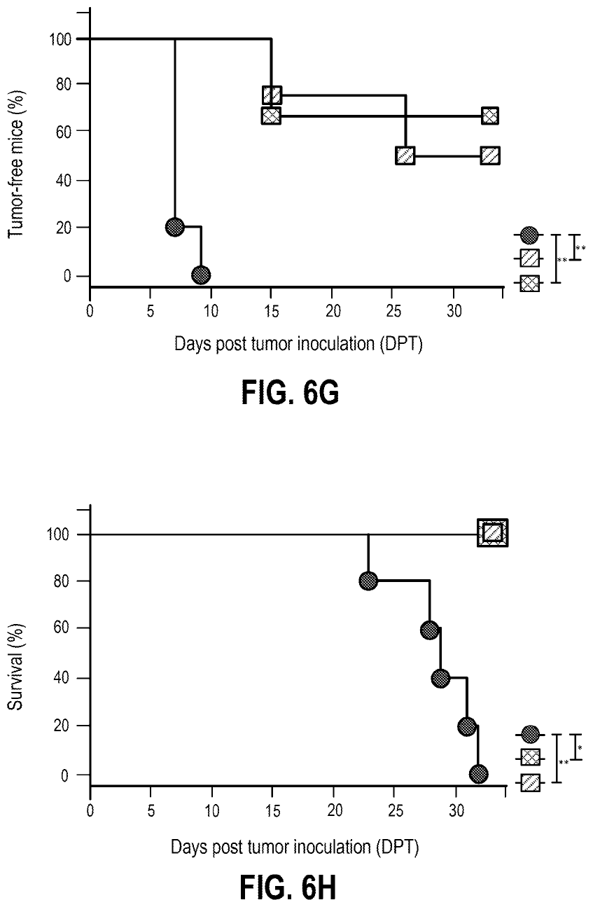

The efficacy of Nano-11 and NanoST as adjuvants for potential therapeutic vaccines was also evaluated. B16-OVA cells were injected subcutaneously (s.c.) into the right flank of C57BL/6J mice. Referring to FIG. 6A, the B16-OVA-bearing mice were vaccinated intradermally with OVA, Nano-11+OVA, or NanoST+OVA at 8 and 15 days post-tumor inoculation. B16-OVA tumors were observed by day 7 in all mice, as FIGS. 6B and 6C illustrate. Referring to FIG. 6D, whereas none of the OVA-vaccinated mice survived to day 20, the survival of the tumor-bearing mice was significantly prolonged by treatment with NanoST+OVA, and the mice had markedly smaller tumors. Referring to FIG. 6E, the identical immunization regimen was administered to E.G7-OVA-bearing mice; however, immunizations were administered at one day and 21 days post-tumor inoculation. Immunization with Nano-11 and NanoST resulted in the inhibition of E.G7-OVA growth, as demonstrated in FIG. 6F. Notably, as FIG. 6G demonstrates, at day 30, 60% and 50% of mice vaccinated with Nano-11 and NanoST, respectively, were free of E.G7-OVA tumors, compared with 100% tumor formation in OVA-vaccinated mice by day 10. Data presented in FIG. 6H establishes that vaccination with Nano-11 and NanoST significantly inhibited the development of E.G7-OVA tumors and resulted in 100% survival compared to OVA-vaccinated mice.

### Example 7—NanoST Stimulates the Differentiation of Antigen-Specific CD8+ T Cells

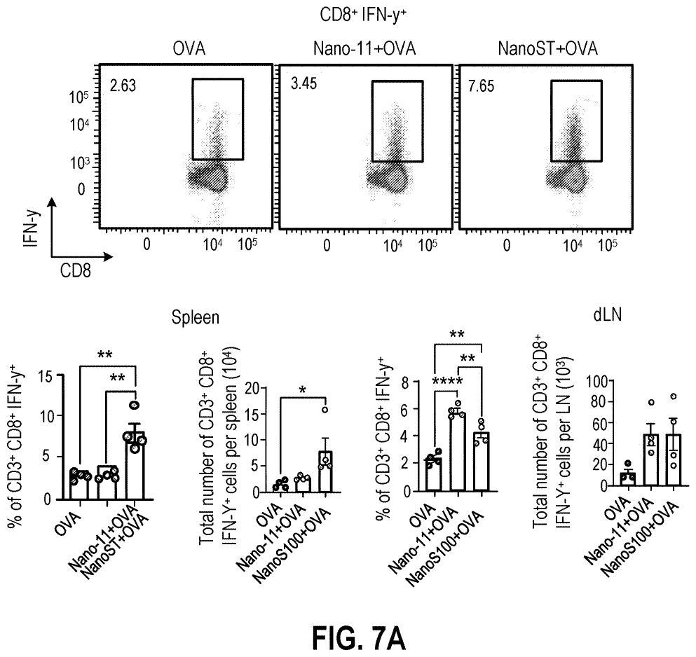

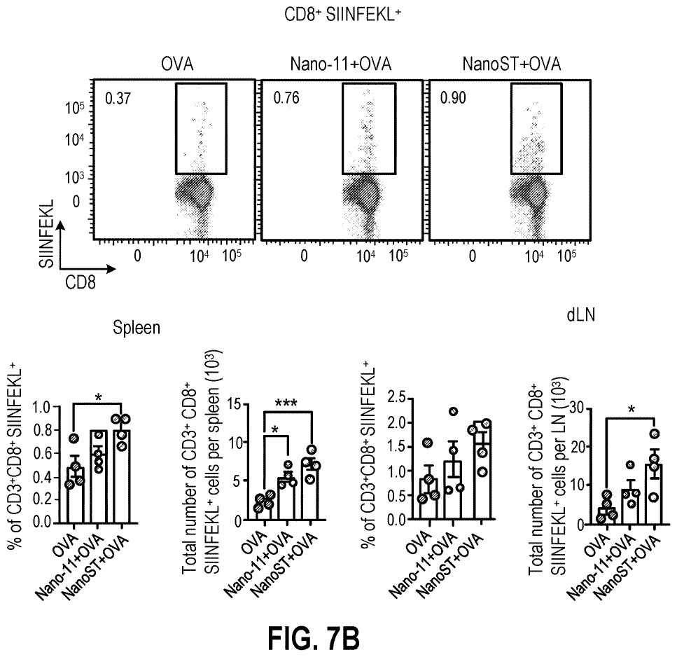

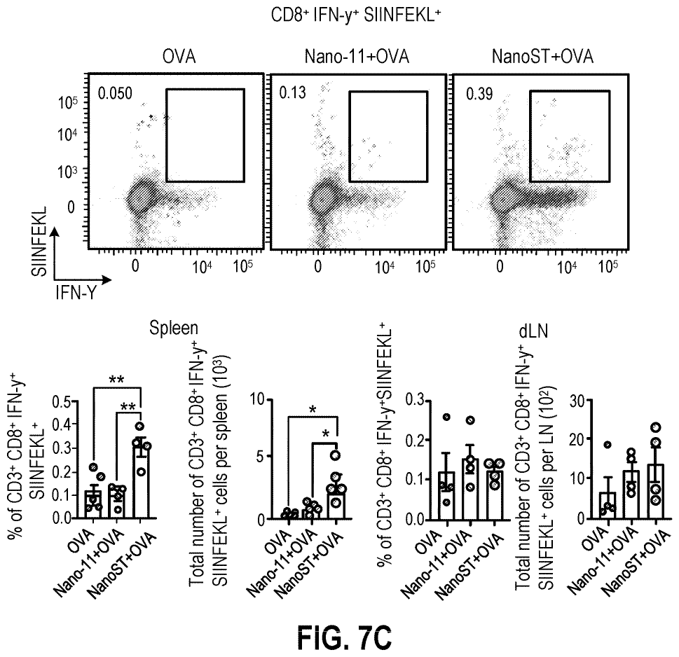

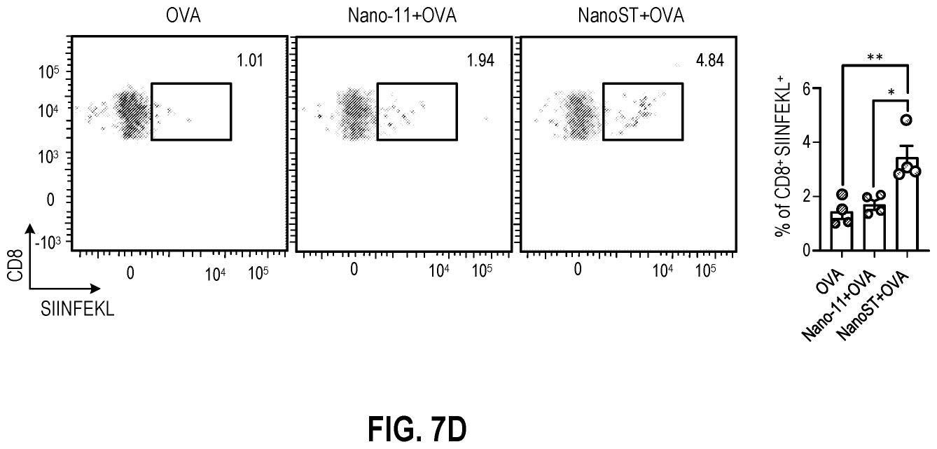

The distribution of OVA-specific CD8+ T cells was determined by utilizing a H-2Kb SIINFEKL-specific MHC class I tetramer. Mice were immunized with OVA, Nano-11+OVA, or NanoST+OVA and 10 days post-secondary vaccination, dLNs, spleen, and bone marrow were collected. Intradermal vaccination with NanoST+OVA led to significant activation of IFN-γ-secreting CD8+ T cells in the spleen (FIG. 7A). However, there was a greater frequency of IFN-γ+ CD8+ T cells in the dLN of mice vaccinated with Nano-11. SIINFEKL+ CD8+ T cells were identified in Nano-11+OVA immunized mice, but the number of these cells was significantly increased in both the spleen and dLN following vaccination with NanoST adjuvant (FIG. 7B). Interestingly, only mice vaccinated with NanoST exhibited an expansion of effector SIINFEKL+ IFN-γ-secreting CD8+ T cells in the spleen (FIG. 7C). Furthermore, the frequency of IINFEKL+ CD8+ T cells was increased in the bone marrow of NanoST+OVA vaccinated mice, indicating their differentiation into effector-memory CD8+ T cells (FIG. 3D). The increased number of SIINFEKL+ IFN-γ+ effector CD8+ T cells suggests that the NanoST+OVA vaccination is facilitating the cross-presentation of OVA and differentiation of antigen-specific CD8+ T cells.

### Example 8—Therapeutic Immunity by Intradermal Vaccination with NanoST Combined with PD-1 Blockade

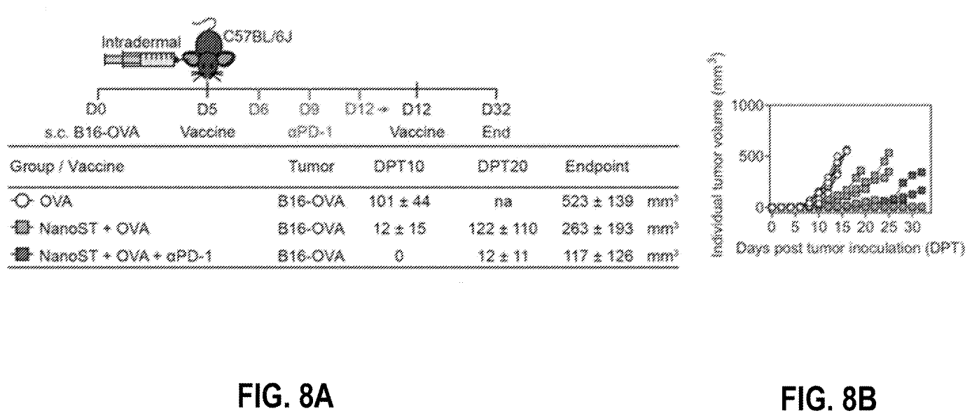

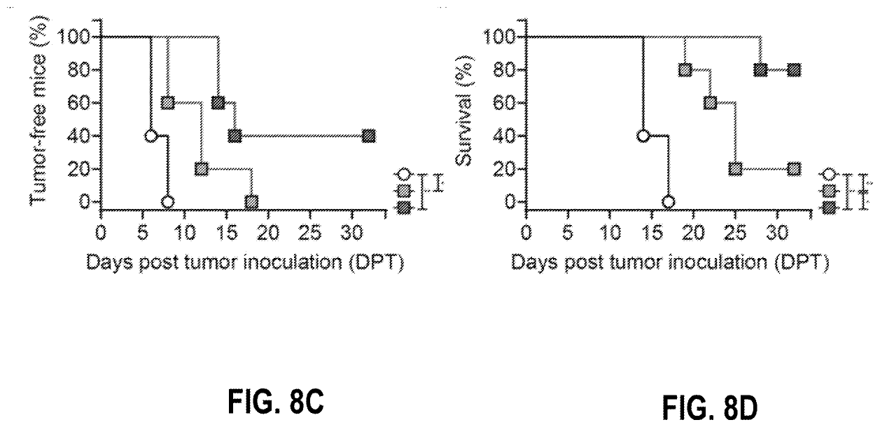

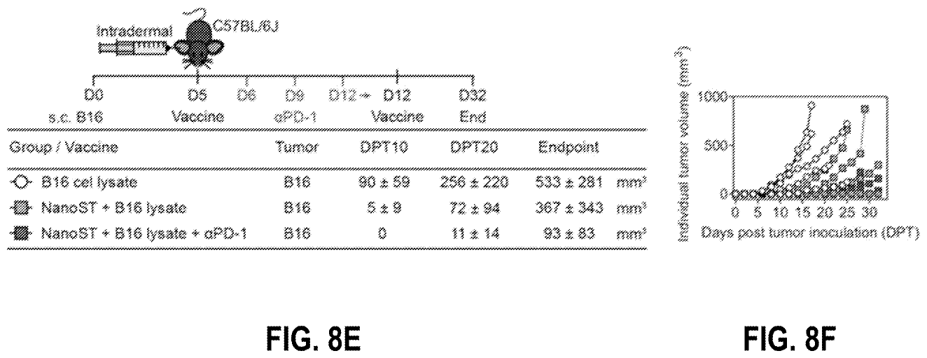

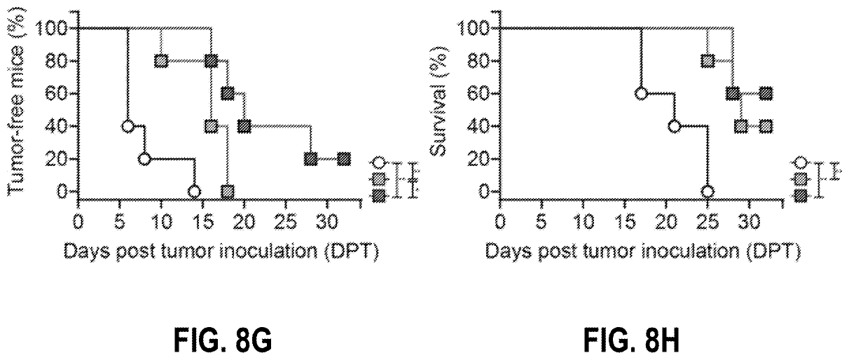

Co-administration of PD-1 checkpoint inhibitor was studied for synergistic effect on the therapeutic efficacy of NanoST. Mice were subcutaneously inoculated with B16-OVA cells, followed by two intradermal vaccinations with NanoST+OVA on day 5 and 12 (FIG. 8A). The treatment of anti-PD-1 was scheduled on day 6, 9, and 12. Combination immunotherapy with PD-1 blockade and NanoST vaccination inhibited the development of the B16-OVA tumors (FIGS. 8B and 8C). These results show that the immunotherapeutic protection induced by intradermal vaccination with OVA and NanoST is further enhanced by the co-administration of the PD-1 checkpoint inhibitor (FIG. 8D). As the identity of tumor antigens in human cancer patients is often unknown, we investigated the immunotherapeutic potential of NanoST-adjuvanted whole cell lysate vaccines. Mice bearing B16 tumors were intradermally vaccinated with NanoST+B16 cell lysate on days 5 and 12 (FIG. 8E). Anti-PD-1 was administered on days 6, 9, and 12. (FIGS. 8F and 8H). The addition of the PD-1 checkpoint blockade synergized with the NanoST+B16 cell lysate vaccine to increase the tumor-free survival rate of mice (FIG. 8F). This data provides evidence for the efficacy and versatility of the NanoST-adjuvant system to intradermally deliver tumor-associated antigens to induce tumor-specific humoral and cell-mediated immunity with both prophylactic and therapeutic protection. Together, this data shows the potential of intradermal vaccination as an innovative avenue for the research and development of next-generation vaccines against both infectious diseases and cancer.

In sum, the present disclosure demonstrates that NanoST-based vaccines facilitate the antigen cross-presentation and differentiation of antigen-specific CD8+ T cells and induce the proliferation of tumor-specific GC B cells and differentiation of Tfh, Th1, and CD8+ IFN-γ+ T cells, resulting in preventive and therapeutic antitumor outcomes.

The stimulator of IFN genes (STING) pathway is an essential component in the activation of antigen-presenting cells (APCs), resulting in enhanced production of type I IFN and proinflammatory cytokines that induce the differentiation of tumor-specific CD8⁺ T lymphocytes. The activation of STING as a cancer therapeutic has paved the way for the development of numerous pharmacologic-class STING agonists that are now in clinical testing (See Amouzegar et al., Cancers 13: 2695(2021)). The first generation of STING agonists were synthetic cyclic dinucleotides (CDNs) like ADU-S100 (MIW815), which were made to be more chemically stable and have a higher affinity for all human STING alleles. Intratumoral injection of ADU-S100 leads to the generation of tumor-specific CD8⁺ T cells in different murine cancer models. Recent findings from two clinical studies that evaluated the efficacy and safety of intra-tumoral injection with ADU-S100 found that there were no dose-limiting toxicities in patients with metastatic solid tumors or lymphomas (Zandberg et al., Annals of Oncology 31: S1446-S1447 (2020), which is incorporated herein by reference; Meric-Bernstam, 2022). Although the studies demonstrated the safety of ADU-S100, they were discontinued because they failed to demonstrate conclusive evidence of anti-tumor activity. Additionally, no adverse reactions were seen when ADU-S100 was used in conjunction with pembrolizumab in a phase II study to treat recurrent or metastatic head and neck squamous cell carcinoma, but this work also demonstrated only modest anti-tumor efficacy (Meric-Bernstam, 2023). The outcomes of these trials confirmed that ADU-S100 is safe; however, the limited anti-cancer efficacy is indicative of suboptimal STING activation by ADU-S100. These conclusions are supported by studies supporting the present disclosure(see, e.g., FIG. 7). This could be caused by CDN-related factors such as in vivo degradation, rapid diffusion from the injection site, or suboptimal intra-tumoral adjuvant delivery.

Considering the clinical outcomes that were reported with STING-targeted activation by ADU-S100, it was discovered that the immunotherapeutic efficacy of ADU-S100 was enhanced when it was delivered intradermally and combined with Nano-11. Previous studies involving swine influenza vaccine adjuvants established the adsorption efficiency of cyclic dinucleotides (CDNs), including ADU-S100, to Nano-11 (see Hernandez-Franco, 2021). Intradermal (i.d.) vaccination with Nano-11-based vaccines increased antigen uptake and transportation by cDC1s and cDC2s into draining lymph nodes. ADU-S100 can be delivered to human monocyte-derived DCs when combined with Nano-11, forming a synergistic combination adjuvant NanoST that enhances the expression of costimulatory CD80 and CD86. The NanoST combination adjuvant also activates the NF-κB and IRF3 signaling cascades that stimulate the expression of type I IFN, proinflammatory cytokines, and chemokines, which are essential for the recruitment and maturation of DCs and their subsequent influence on the differentiation of CD4⁺ and CD8⁺ T cells. Type I IFN derived from STING-activated DCs may produce IL-6, which can facilitate the differentiation of T follicular helper (Tfh) cells and signal germinal center (GC) B cell proliferation. Previous work in IFN-α or IFN-γ receptor knockout mice reported a reduction in the differentiation of Th1 and Tfh cells, in addition to a loss of GC B cell responses that resulted in the inhibition of IgG2c class switching. Thus, activation of STING can also influence the differentiation and function of Tfh cells and GC B cells, which are critical for the generation of high-affinity antibodies that can contribute to cancer control. In fact, Tfh and GC B cell differentiation are associated with long-term survival in patients with colorectal or breast cancer. The present disclosure demonstrates that intradermal vaccination with Nano-11 and NanoST enriched the population of GC B and Tfh cells in the draining lymph nodes and increased the generation of antigen-specific IgG1, Ig2c, and long-lived plasma cells. These findings suggest potential connections between STING-mediated activation of DCs and their subsequent mobilization of T-cell-mediated immunity.

The present disclosure further demonstrates that cDC1 cells are activated by intradermal vaccination with Nano-11 and CDNs, confirming cDC1s engagement in the STING-mediated immune response. This is a promising adjuvant function of Nano-11, since cDC1s are involved with the polarization of CD4⁺ T cells towards the IFN-γ secreting Th1 effector subset, which enhances the activity of CTLs. Human and murine cDC1s have been associated with superior cross-presentation of antigens, which results in the activation of antigen-specific cytotoxic T-cell immunity, further supporting the development of STING-targeted therapies. Additionally, tumor eradication correlates with the polarization and proliferation of Th1 cells and the activation of CD8⁺ cytotoxic T lymphocytes (CTLs), rather than the expansion of Th17 lineage cells. This is in line with a previously reported study demonstrating enhanced differentiation of Th1 and CD8⁺ IFN-γ⁺ T cells following intradermal vaccination with NanoST, as well as vaccine antigen uptake by cDCIs and induction of Th1 cells (Hernandez-Franco, 2021). Moreover, the multifunctional anti-cancer effects of the type I IFN system upon STING activation include the enhancement of antigen presentation and cross-presentation to CD8⁺ T cells through cDC1-mediated cross-priming. This is consistent with the in vitro and in vivo cytotoxicity CD8⁺ T cell experiments, which demonstrate that the synergy of the NanoST combination adjuvant is attributable to the potent induction of cross-presentation and activation in DCs, which in turn initiate the antigen-specific target lysis of cells by cytotoxic CD8⁺ T cells. Finally, the data show that NanoST is effective at inducing tumor-specific CTLs, as evidenced by the cross-presentation of vaccine antigen, which elicited both prophylactic and therapeutic-mediated protection against murine cancer models of melanoma and lymphoma. These findings are consistent with other investigations showing that DCs efficiently uptake antigen and orchestrate the activation of tumor-specific CTLs and Th1 cells after intradermal vaccination (Mitsui et al., British Journal of Dermatology 162: 29-41 (2010); Inoue et al., Cancer Science 114: 34-47 (2023)). In addition, clinical success has been associated with the presence of GC B cells, Tfh, Th1, and CD8⁺ effector T cells in human lung adenocarcinomas. Thus, the participation of all these immune cells is essential for the establishment of an effective anti-cancer immune response.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure. The term “or” is used to refer to a nonexclusive “or” unless otherwise indicated.

Unless defined otherwise, all technical and scientific terms used herein have the same meaning as commonly understood to one of ordinary skill in the art. The following terms and phrases shall have the meaning indicated.

The term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value, such as within 10%, within 5%, or within 1% of a stated value or stated limit of a range) provided that one of ordinary skill in the art would consider equivalent to the recited value (e.g., having the same function or result). The term “substantially” can allow for a degree of variability in a value or range, for example, within 90%, within 95%, 99%, 99.5%, 99.9%, 99.99%, or at least about 99.999% or more of a stated value or of a stated limit of a range.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

What is claimed is:

1. A method of prophylactically or therapeutically vaccinating a subject for cancer, which method comprises administering to the subject an immune response-inducing effective amount of a composition comprising an agonist of the stimulator of interferon (IFN) genes (STING) and a tumor (or cancer) antigen, both of which are adsorbed onto cationic phytoglycogen (PG) nanoparticles, whereupon the subject is vaccinated for cancer.

2. The method of claim 1, wherein the cationic PG nanoparticles are prepared by conjugating PG with octenyl succinic anhydride (OS) and (3-chloro-2-hydroxypropyl)-trimethylammonium chloride (CHPTAC) (PG-OS-CHPTAC (also known as Nano-11)).

3. The method of claims 1 or 2, wherein the adsorption of the STING agonist onto cationic PG nanoparticles results in a synergistic effect.

4. The method of claim 3, wherein the synergistic effect comprises an enhanced CD4+ and CD8+ T cell response via activation of NF-κB and IRF3 signaling pathways.

5. The method of claim 2, wherein the cationic PG nanoparticles are derived from sweet corn.

6. The method of claim 5, wherein the sweet corn encodes the sugary-1 mutant gene.

7. The method of claim 1, wherein the STING agonist is a cyclic dinucleotide (or analogue thereof) or a cyclic mononucleotide (or analogue thereof).

8. The method claim 7, wherein the STING agonist is ADU-S100 (also known as MIW815).

9. The method claim 1, wherein the STING agonist is a noncyclic dinucleotide (or analogue thereof) or a noncyclic mononucleotide (or analogue thereof).

10. The method of claim 1, wherein the STING agonist is an amidobenzimidazole or a dimeric amidobenzimidazole.

11. The method of claims 1, wherein the STING agonist is selected from the group consisting of SYNB1891, MK-1454 (which is available from Merck), MK-2118, BMS-986301, SR-717, GSK3745417, SB-11285 (which is available from Spring Bank Pharmaceuticals), AdVCA0848, STINGVAX, IMSA-101, c-di-GMP (e.g., 2′3′-c-diGMP or halogenated c-di-GMP, such as fluorinated), c-di-AMP (e.g., 2′3′-c-di-AM(PS)2 (Rp, Rp) or halogenated c-di-AMP, such as fluorinated), cGAMP (e.g., 2′2′-cGAMP, 2′3′-cGAMP, 3′3′-cGAMP, 2′3′-cGAM(PS)2 (Rp/Sp), or halogenated 3′3′-cGAMP, such as fluorinated), cAIMP (e.g., cAIMP Difluor or cAIM(PS)2 Difluor (Rp/Sp)), cAMP-GMP, c-AMP-IMP, cGMP-IMP, 2′-5′, 3′-5′ mixed phosphodiester linkage (ML) RR-S2 c-di-AMP (also known as ML RR-S2 CDA), ML RR-S2-c-di-GMP (also known as ML-CDG), ML RR-S2 cGAMP, c-di-IMP, and vadimezan (also known as ASA-404 and DMXAA).

12. The method of claim 1, wherein the tumor antigen is a tumor-associated antigen (TAA).

13. The method of claim 1, wherein the tumor antigen is a tumor-specific antigen (TSA).

14. The method of claim 13, wherein the tumor antigen is a neoantigen.

15. The method of claim 11, wherein the tumor antigen is selected from the group consisting of CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRyll1); ganglioside G2 (GD2); ganglioside GD3 (aNeuSAc(2-8)aNeuSAc(2-3)bDGaip(1-4)bDGIcp(1-1)Cer), TNF receptor family member B cell maturation (BCMA); Tn antigen ((Tn Ag) or (GalNAcu-Ser/Thr)); prostate-specific membrane antigen (PSMA); Receptor tyrosine kinase-like orphan receptor 1 (RORI); Fms-Like, Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21(Testisin or PRSS 21); vascular endothelial growth factor receptor 2 (VEGFR2); Lewis(Y)antigen; CD24; Platelet-derived growth factor receptor beta (PDGFR-beta); Stage-specificembryonic antigen-4 (SSEA-4); CD20; delta like 3 (DLL3); Folate receptor alpha; Receptor tyrosine-protein kinase, ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2(EphA2); Fucosyl GM1; sialyl Lewis adhesion molecule (sLe); ganglioside GM3 (aNeuSAc(2-3)bDGalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); six transmembrane epithelial antigen of the prostate I (STEAP1); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (ORS IE2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MADCT-1); melanoma cancer testis antigen-2 (MAD-CT-2); Fos-related antigen 1; tumor protein p53, (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1(CYP IBI); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES I); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-I); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1); and neoantigens.

16. The method of claim 11, wherein the tumor antigen is selected from the group consisting of CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37,CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NYESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-I, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D 1), ephrinB2, epithelial tumor antigen, estrogen receptor, fetal acetylcholine receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-microglobulin, and Fc Receptor-like 5 (FcRL5).

17. The method of claim 13, wherein the administering is intradermally.

18. The method of claim 17, wherein the administering involves the use of a microneedle injector, microneedle patch, or needle-free injector.

19. The method of either of claims 12 or 13, wherein the cancer is melanoma.

20. The method of either of claims 12 or 13, wherein the cancer is bladder, breast, cervical, colon, colorectal, colorectal adenocarcinoma, gastroesophageal, gastrointestinal stromal, hepatocellular, kidney, leukemia, lymphoma, malignant mesothelioma, multiple myeloma, myelodysplastic syndrome, neuroblastoma, non-small cell lung, ovarian, pancreatic, plasma cell neoplasms, prostate, sarcoma, small cell lung, testicular, transitional cell carcinoma, urothelial, or Wilm's tumor.

21. A microneedle device comprising a plurality of microneedles onor in which are contained cationic phytoglycogen (PG) nanoparticles onto which are adsorbed (i) an agonist of the stimulator of interferon (IFN) genes (STING) and (ii) a tumor (or cancer) antigen or a neoantigen.

22. The microneedle device of claim 22, which is a microneedle patch.

23. A needle-free injector containing a composition comprising cationic phytoglycogen (PG) nanoparticles onto which are adsorbed (i) an agonist of the stimulator of interferon (IFN) genes (STING) and (ii) a tumor (or cancer) antigen or a neoantigen.

24. A kit comprising a microneedle device and a composition comprising (or consisting essentially of or consisting of) cationic phytoglycogen (PG) nanoparticles onto which are adsorbed (i) an agonist of the stimulator of interferon (IFN) genes (STING) and (ii) a tumor (or cancer) antigen or a neoantigen.

25. A kit comprising a needle-free injector and a composition comprising cationic phytoglycogen (PG) nanoparticles onto which are adsorbed (i) an agonist of the stimulator of interferon (IFN) genes (STING) and (ii) a tumor (or cancer) antigen or a neoantigen.

26. A pharmaceutical composition for intradermal immunization against a cancer, the composition comprising a NanoST nanoparticle adjuvant and a cancer antigen adsorbent.

27. The pharmaceutical composition of claim 26, wherein the cancer is a melanoma.

28. The pharmaceutical composition of claim 27, wherein the cancer antigen is a tumor-associated antigen (TAA), characterized by overexpression in melanoma cells.

29. The pharmaceutical composition of claim 28, wherein the TAA is one of tyrosinase, MAGE (melanoma antigen gene)-A1, -C1, and -C2, Melan-A, Gp100, TRP-1, TRP-2, MC1R, NY-ESO-1, and SSX-2.

30. The pharmaceutical composition of claim 27, wherein the cancer antigen is a tumor-specific antigen (TSA), wherein the TSA comprises an epitope or determinant that constitutes or incorporates a mutation-derived biomarker specific to melanoma.

31. A method of administering a combination immunotherapy, wherein the method comprises:
administering to a subject an immune response-inducing effective amount of a cancer vaccine comprising a NanoST nanoparticle adjuvant and a cancer antigen adsorbent; and
co-administering to the subject a blockade-inducing effective amount of a PD-1 checkpoint inhibitor; whereupon, through stimulatory activation of NF-κB and IRF3 signaling pathways induced through administration of the cancer vaccine and a checkpoint protein blockade induced through co-administration of the immune checkpoint inhibitor, the subject is immunized against a cancer.

32. The method of claim 31, wherein administration and co-administration are performed according to a preventative immunotherapy schedule comprising:
administration of a first dose of the cancer vaccine on D1;
co-administration of the immune check point inhibitor according to a dosing regimen beginning any one of days D3-D6; and
administration of a second dose of the cancer vaccine between D14 and D21.

33. The method of claim 31, wherein administration and co-administration are performed according to a therapeutic immunotherapy schedule comprising:
administration of a first dose of the cancer vaccine on a first day (D1);
co-administration of the PD-1 immune check point inhibitor according to a dosing regimen beginning on any one of days D3 or D4; and
administration of a second dose of the cancer vaccine between D5 and D8.

34. The method of either of claims 32 or 33, wherein the dosing regimen is a single dose regimen.

35. The method of either of claims 32 or 33, wherein the dosing regimen is a multiple dose regimen.
